SEQUENCE LISTING (60 sequences)

SEQ ID NO: 1 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEVSGTPLYAGKNVLDNSKGTMDQELLTPEFNYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGVNIGAVLTKCQQKGWGDFRNFQPSGRDVIVKGQGTFTSNYGTDFILKIEDITDSKLRNNNGSGTVVQEIKVPLIRTEI

SEQ ID NO: 2 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEVSGTPLYAGKNVLDNSKGTMDQELLTPEFNYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGINIGAVLTKCQQKGWGDFRNFQPSGRDVIVKGQGTFKSNYGTDFILKIEDITDSKLRNNNGSGTVVQEIKVPLIRTEI

SEQ ID NO: 3 - protein, 295 aa, synthetic construct
MSSTDVQERLRDLARENEAGTLNEAWNTNFKPSDEQQFSYSPTEGIVFLTPPKNVIGERRISQYKVSNAWATLEGSPTEVSGTPLYAGKNVLDNSKGTMDQELLTPEFNYTYTESTSNTTTHGLKLGVKTTATMKFPIAQGSMEASTEYNFQNSSTDTKTKQVSYKSPSQKIKVPAGKTYRVLAYLNTGSISGEANLYANVGGIAWGVLPGYPNGGGINIGAVLTKCQQKGWGDFRNFQPSGRDVIVKGQGTFKSNYGTDFILKIEDITDSKLRNNNGSGTVVQEIKVPLIRTEI

SEQ ID NO: 4 - protein, 295 aa, synthetic construct
MSSTDVQERLRDLARENEAGTLNVAWNTNFKPSDEQQFSYSPTEGIVFLTPPKNVIGERRISQYKVNNAWATLEGSPTEVSGTPLYAGKNVLDNSKGTMDQELLTPEFNYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGINIGAVLTKCQQKGWGDFRNFQPSGRDVIVKGQGTFKSNYGTDFILKIEDITDSKLRNNNGSGTVVQEIKVPLIRTEI

SEQ ID NO: 5 - protein, 295 aa, synthetic construct
MSSTDVQERLRDLARENEAGTLNVAWNTNFKPSDEQQFSYSPTEGIVFLTPPKNVIGERRISQYKVNNAWATLEGSPTEVSGTPLYAGKNVLDNSKGTMDQELLTPEFNYTYTESTSNTTTHGLKLGVKTTATMKFPIAQGSMEASTEYNFQNSSTDTKTKQVSYKSPSQKIKVPAGKTYRVLAYLNTGSISGEANLYANVGGIAWGVLPGYPNGGGINIGAVLTKCQQKGWGDFRNFQPSGRDVIVKGQGTFKSNYGTDFILKIEDITDSKLRNNNGSGTVVQEIKVPLIRTEI

SEQ ID NO: 6 - protein, 295 aa, synthetic construct
MSSTDVQERLRDLARENEAGTLNVAWNTNFKPSDEQQFSYSPTEGIVFLTPPKNVIGERRISQYKVNNAWATLEGSPTEVSGTPLYAGKNVLDNSKGTSDQELLTPEFNYTYTESTSNTTTHGLKLGVKTTATMKFPIAQGSMEASTEYNFQNSSTDTKTKQVSYKSPSQKIKVPAGKTYRVLAYLNTGSISGEANLYANVGGIAWGVLPGYPNGGGINIGAVLTKCQQKGWGDFRNFQPSGRDVIVKGQGTFKSNYGTDFILKIEDITDSKLRNNNGSGTVVQEIKVPLIRTEI

SEQ ID NO: 7 - protein, 295 aa, synthetic construct
MSSTDVQERLRDLARENEAGTLNEAWHTNFKPSDEQQFSYSPTEGIVFLTPPKNVIGERRISQYKVNNAWATLEGSPTEVSGTPLYAGKNVLDNSKGTMDQELLTPEFNYTYTESTSNTITHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGVNIGAVLTKCQQKGWGDFRNFQPSGRDVIVKGQGTFESNYGTDFILKIEDITDSKLRNNNGSGTVVQEIKVPLIRTEI

SEQ ID NO: 8 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEMSGTPLYAGKNVLDNSKGTSDQELLTPEFNYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGVNIGAVLTKCQQKGWGDFRNFQPSGRDVIVKGQGTFTSNYGTDFILKIEDITDSKLRNNNGSGTVVQEIKVPLIRTEI

SEQ ID NO: 9 - protein, 295 aa, synthetic construct
MSSTDVQERLRDLAREDEAGTFNEAWNTNFKPSDEQQFSYSPTEGIVFLTPPKNVIGERRISQYKVNNAWATLEGSPTEVSGTPLYVGKNVLDNSKGTSDQELLTPEFNYTYTESTSNTTTHGLKLGVKTTATMKFPIAQGSMEASTEYNFQDSSTDTTTKTVSYKSPSQKIKVPAGKTFRVLAYLNTGSISGEANLYANVGGIAWGVLPGYPNGGGVNIGAVLTKCQQKGWGEFRNFQPSGRDVIVKGQGTFKSNYGTDFILKIEDITDSKLRNNNGSGTVVQEIKVPLIRTEI

SEQ ID NO: 10 - protein, 295 aa, synthetic construct
MSSTDVQERLRDLARENEAGTLNVAWNTNFKPSDEQQFSYSPTEGIVFLTPPKNVIGERRISQYKVNNAWATLEGSPTEVSGTPLYAGKNVLDNSKGTSDQELLTPEFNYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGINIGAVLTKCQQKGWGDFRNFQPSGRDVIVKGQGTFKSNYGTDFILKIEDITDSKLRNNNGSGTVVQEIKVPLIRTEI

SEQ ID NO: 11 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEMSGTPLYAGKNVLDNSKGTSDQELLTPEFTYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWGVLPGYPNGGGVNIGAVLTKCQQKGWGDFRNFQPSGRDVIVKGQGTFTSNYGTDFILKIEDITDSKLRNNNGSGTVVQEIKVPLIRTEI

SEQ ID NO: 12 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEVSGTPLYAGKNVLDNSKGTSDQELLTPEFNYTYTESTSNTTTHGLKLGVKTTATMKFPIAQGSMEASTEYNFQDSSTDTTTKTVSYKSPSQKIKVPAGKTFRVLAYLNTGSISGEANLYANVGGIAWGVLPGYPNGGGVNIGAVLTKCQQKGWGDFRNFQPSGRDVIVKGQGTFKSNYGTDFILKIEDITDSKLRNNNGSGTVVQEIKVPLIRTEI

SEQ ID NO: 13 - protein, 295 aa, synthetic construct
MSSTDVQERLRDLARENEAGTLNVAWNTNFKPSDEQQFSYSPTEGFIFLTPPKNVIGERRISHYKVNNAWATLEGSPTEVSGTPLYAGRNVLDNSKGTIDQEMLTPEFNYTYTEGTSNTTTHGLKLGVKTTATMKFPIAQGSMEASTEYNFQNSSTDTKTKQVSYKSPSQKIKVPAGKTFRVLAYLNTGSISGEANLYANVGGVAWGVLPGYPNGGGVNIGAVLTKCQQKGWGDFRNFQPSGRDVIVKGQGTFTSNYGTDFILKIEDITDSKLRNNNGSGTVVQEIKVPLIRTEI

SEQ ID NO: 14 - protein, 295 aa, synthetic construct
MSSTDVQERLRDLAREDEAGTFNEAWNANFKPSDEPQFSNSPTQGIVILAPPKNVIGERRISQYKVNNAWATLEGSPTEVSGTPLYAGRNVLDNSKGTMDQELLTPEFTYTYTESTSNTTTHGLKLGVKTTATMKFPIAQGSMEASTEYNFQNSSTDTKTKQVSYKSPSQKIKVPAGKTFRVLAYLNTGSISGEANLYANVGGIAWRVSPGYPNGGGVNIGAVLTKCQQKGWGDFRNFQPSGRDVIVKGQGTFKSDYGTDFILKIEDITDSKSRNNNGSGTVVQEIKVPLIRTEI

SEQ ID NO: 15 - protein, 295 aa, synthetic construct
MSSTDVQERLRDLARENEAGTLNVAWNTNFKPSDEQQFSYSPTEGFIFLTPPKNVIGERRISHYKVNNAWATLEGSPTEVSGTPLYAGRNVLDNSKGTIDQEMLTPEFNYTYTEGTSNTTTHGLKLGVKTTATMKFPIAQGSMEASTEYNFQNSSTDTKTKQVSYKSPSQKIKVPAGKTFRVLAYLNTGSISGEANLYANVGGVAWGVLPGYPDGGGVNIGAVLTKCQQKGWGDFRNFQPSGRDVIVKGQGTFTSNYGTDFILKIEDITDSKLRNNNGSGTVVQEIKVPLIRTEI

SEQ ID NO: 16 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEVSGTPLYAGKNVLDNSKGTMDQELLTPEFNYTYTESTSNTTTHGLKVGVKTTATMKFPIAQGSMEASTEYNFQDSSTDTTTKTVSYKSPSQKIKVPAGKTFRVLAYLNTGSISGEANLYANVGGIAWGVLPGYPNGGGVNIGAVLTKCQQKGWGDFRNFQPSGRDVIVKGQGTFKSNYGTDFILKIEDITDSKLRNNNGSGTVVQEIKVPLIRTEI

SEQ ID NO: 17 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEVSGTPLYVGKNVLDNSKGTMDQELLTPEFNYTYTESTSNTTTHGLKLGVKTTATMKFPIAQGSMEASTEYNFQDSSTDTTTKTVSYKSPSQKIKVPAGKTFRVLAYLNTGSISGEANLYANVGGIAWGVLPGYPNGGGVNIGAVLTKCQQKGWGDFRNFQPSGRDVIVKGQGTFKSNYGTDFILKIEDITDSKLRNNNGSGTVVQEIKVPLIRTEI

SEQ ID NO: 18 - protein, 295 aa, synthetic construct
MSSTDVQERLRDLARENEAGTLNEAWNTNFKPSDEQQFSYSPTEGIVFLTPPKNVIGERRISQYKVNNAWATLEGSPTEVSGTPLYVGKNVLDNSKGTMDQELLTPEFNYTYTESTSNTTTHGLKVGVKTTATMKFPIAQGSMEASTEYNFQDSSTDTTTKTVSYKSPSQKIKVPAGKTFRVLAYLNTGSISGEANLYANVGGIAWGVLPGYPNGGGVNIGAVLSKCQQKGWGDFRNFQPSGRDVIVKGQGTFKSNYGTDFILKIEDITDSKLRNNNGSGTVVQEIKVPLIRTEI

SEQ ID NO: 19 - DNA, 888 bp, synthetic construct
  source 1..888: mol_type = other DNA
atgagttcaacagatgtacaagaaaggctacgcgacctcgcgcgtgaaaacgaggccgggaccttgaatgaggcttggaataccaatttcaaaccgtccgatgaacagcagtttagctacagcccgactgagggtatcgtgtttctgacacctccgaaaaacgtgatcggtgagcgccgtattagccaatataaagtcaacaacgcgtgggcaacgctggaaggttccccgaccgaggtttcaggtacgccattgtacgcgggtaagaacgtcctggataacagcaagggtacgatggatcaagagctgctgaccccggaatttaactacacctataccgaatcgacttctaataccacaacccacggcctgaaactgggtgttaagaccactgccaccatgaagtttccgattgcacagggtagcatggaggcgagcacggagtataacttccagaattcctctacggacaccaagaccaagcaggttagctacaagagcccgtcgcaaaaaatcaaggtgccggcaggtaaaacgttccgtgttttggcgtatctgaacaccggtagcattagcggcgaggcgaatctgtacgcgaacgtaggtggcattgcgtggggcgtcttaccgggttatccgaatggcggtggcgtgaacattggtgctgtgttgaccaagtgccagcaaaaaggctggggtgatttccgcaacttccaaccgtctggccgtgacgtgattgttaaaggccaaggcaccttcacctccaattacggcactgactttatcctgaagattgaagatatcaccgacagcaaactgcgtaataacaacggcagcggcaccgttgttcaggagatcaaagtgccacttatcagaaccgaaatctaa

SEQ ID NO: 20 - DNA, 888 bp, synthetic construct
  source 1..888: mol_type = other DNA
atgagttcaacagatgtacaagaaaggctacgcgacctggcgagagaaaatgaagcgggtacgttaaacgaggcttggaataccaatttcaaaccgagcgatgaacaacaatttagctacagcccgaccgagggcatcgtgtttctgaccccgcctaaaaacgtcatcggcgaacgtcgtatcagccagtataaagtcaacaacgcgtgggcaaccctcgaaggcagcccaacggaggtcagcggcaccccgttgtacgctggcaagaacgtgctggataatagcaaaggcaccatggatcaagagttgctgaccccggaatttaattacacctatactgagagcacctcaaacaccactacgcacggcctgaaactgggtgttaaaaccaccgcaaccatgaaatttccgattgctcagggttccatggaagcgagtaccgagtataacttccaaaactccagcacggacaccaagaccaagcaggtttcctacaaaagcccgtcgcaaaaaatcaaggtgccggcaggcaagaccttccgcgtattggcgtatctgaataccggttccatttctggcgaggcgaacctgtacgcgaatgtgggtggtatcgcctggggtgtgctgccgggctatccgaatggtggtggcattaacatcggtgccgttttgacgaagtgccagcagaagggttggggtgattttcgtaacttccagccgtctggtcgtgacgttattgtgaagggtcagggtactttcaagtcgaactacggcacggacttcatcctgaagatcgaggatattaccgacagcaaactgcgtaacaacaacggctctggtacagttgttcaggagatcaaagtgccacttattcgcaccgaaatttaa

SEQ ID NO: 21 - DNA, 888 bp, synthetic construct
  source 1..888: mol_type = other DNA
atgagttcaacagatgtacaagaaaggctacgcgacctggcgcgtgaaaacgaggcgggcacgttgaacgaggcgtggaataccaattttaaaccgtcggatgaacagcagtttagctacagcccgacagagggtatcgtgtttctgaccccgcctaaaaacgtgatcggtgaacgccgtatcagccagtataaggtgtctaacgcttgggcaaccctcgaaggcagcccgaccgaggtctccggcactccattgtacgcgggaaagaacgtgttggataattctaagggcaccatggatcaagagctgctgactccagaatttaactacacctacaccgagagcaccagcaacactaccacccacggcctgaagctgggtgtgaagacgaccgcaaccatgaaattcccgattgcgcagggttccatggaagcgagcacggaatataacttccaaaacagcagcaccgataccaagacgaagcaggttagctacaaatccccgtcgcaaaaaatcaaagttccggcaggtaagacctatcgtgttcttgcctatctgaatacgggtagtatcagcggtgaggcgaacctgtatgctaatgttggtggcatcgcctggggtgttctgccgggttacccgaatggtggtggtattaatattggcgctgtgttgaccaagtgccagcaaaaaggttggggcgacttccgcaacttccaaccgtctggccgtgacgttattgtgaagggtcagggtacgtttaaatccaactacggcaccgacttcatcctgaaaattgaagatattaccgacagcaaattacgtaataacaacggctccggcactgtagttcaggagatcaaggtcccgctgattcgtaccgagatctaa

SEQ ID NO: 22 - DNA, 888 bp, synthetic construct
  source 1..888: mol_type = other DNA
atgagttcaacagatgtacaagaaaggctacgcgacctggcacgtgaaaacgaagctggcaccctcaacgttgcgtggaacacgaacttcaaaccgagcgatgaacagcaatttagctacagcccgaccgagggcattgtttttctgacgccgccaaaaaacgtgattggtgaacgtcgtattagccagtataaagtgaataatgcttgggcaaccttggaaggtagtccaactgaggttagcggcaccccgctgtacgccggtaagaacgttttagataatagcaagggcacaatggatcaagagttgctgaccccggaatttaactatacctacaccgagagcaccagcaacaccacgacccacggcctgaaacttggtgttaaaaccaccgcgaccatgaaattcccgattgcgcagggctcgatggaagcgagcaccgagtataacttccaaaactccagcactgacaccaagactaagcaggtgtcttacaagagcccgtcgcaaaaaatcaaggtgcctgcaggcaagacctttcgtgttttggcgtatctgaataccggttccatctctggcgaggccaatctgtatgcgaacgtgggtggcatcgcgtggggtgtgctgccgggttacccgaatggcggtggtatcaacattggcgctgtgttgaccaagtgccaacagaagggatggggtgatttccgcaactttcaaccgtccggccgtgacgtcatcgtgaaaggtcagggtacgttcaaatccaattacggtacggacttcatcctgaagatcgaagatattaccgacagcaaactgagaaataacaacggttctggtactgtagtccaggagatcaaagttccgctgattcgcaccgagatctaa

SEQ ID NO: 23 - DNA, 888 bp, synthetic construct
  source 1..888: mol_type = other DNA
atgagttcaacagatgtacaagaaaggctacgcgacctggcgcgtgaaaatgaagcgggcacgctgaatgtcgcttggaacacgaacttcaagccgagcgatgaacagcaattttcctacagcccaactgaaggtattgtgtttctgaccccaccgaaaaacgttatcggcgagcgccgtatttcccaatataaggttaataacgcgtgggcaacattggaaggttccccgaccgaggtgagtggcaccccgctgtacgctggcaagaacgtgttggacaacagcaaaggcaccatggatcaagagttgctgaccccggaattcaattacacctacaccgagagcaccagcaacactacgacccacggtctgaagctgggtgtgaaaaccaccgcaaccatgaaatttccgattgcccagggtagcatggaagcgagcacggagtataatttccaaaactcgtcaaccgataccaagaccaagcaggtttcctacaagagcccgtctcaaaaaattaaagtaccggcaggcaagacctatcgtgttttagcgtatctgaacaccggtagcatttctggcgaggcgaacctgtacgccaatgtgggtggcatcgcgtggggagtgctcccgggttatccgaatggtggcgggatcaacattggcgctgtgttgacgaagtgccagcagaaaggttggggtgatttccgtaatttccagccgagtggtcgtgacgttatcgttaaaggccaaggtacgtttaaaagcaattacggtactgacttcatcctgaaaatcgaagatatcaccgacagcaaactaagaaacaacaacggttctggcaccgttgtccaggagatcaaggtcccgcttatccgcaccgagatttaa

SEQ ID NO: 24 - DNA, 888 bp, synthetic construct
  source 1..888: mol_type = other DNA
atgagttcaacagatgtacaagaaaggctacgcgacttagcgcgtgaaaatgaagcgggtacgttgaacgtggcctggaataccaacttcaaaccgtccgacgaacagcagttttcttacagcccgacggagggtattgtgtttctgactccaccgaaaaacgtgatcggtgagcgccgtattagccagtataaggtaaataacgcttgggcaacccttgagggctcgcctaccgaggtttctggtacgccgctgtatgcgggcaaaaacgtgctggataacagcaaaggcacttcagatcaggagctgctgaccccggaattcaactacacgtacaccgaatctacctccaacaccaccacgcacggcctgaaactgggtgttaagactaccgcgaccatgaaattcccgattgcacagggttccatggaagcgagcaccgagtataacttccaaaactcaagcactgacaccaagaccaagcaggttagctacaagagcccatcgcaaaaaatcaaggttccggcaggcaaaacctaccgtgtcttagcctatctgaataccggttccattagcggcgaggcgaatctgtacgcgaacgttggtggcatcgcttggggggtgttgccgggttatccgaatggaggcggtatcaacattggcgctgtgttgacgaagtgccagcaaaaaggttggggcgattttagaaactttcagccgtccggtcgtgatgttattgtgaagggccaaggcacctttaagagcaattacggcaccgacttcatcctgaagatcgaagatatcaccgacagcaaattgcgcaacaacaatggtagcggtacagtcgttcaagagatcaaagtgccgctgattcgtaccgaaatctaa

SEQ ID NO: 25 - DNA, 888 bp, synthetic construct
  source 1..888: mol_type = other DNA
atgagttcaacagatgtacaagaaaggctacgcgacttggcgcgtgaaaacgaggcgggcaccttgaacgaggcctggcacaccaatttcaaaccgagcgatgaacagcagtttagctacagcccgaccgagggtattgtctttctgaccccgccaaagaatgtcattggcgaacgtcgtatctcgcaatataaagttaataacgcgtgggcaaccttggaaggcagtccgaccgaagtgagcggcactccgctgtacgcaggcaaaaacgtgctggacaacagcaaaggcaccatggatcaggagctgctgactccggaattcaactacacctataccgagtctacgtcaaataccattacccatggtctgaagctcggcgtgaagacgacggcaaccatgaaatttccgattgctcaaggcagcatggaagcgagcaccgagtataacttccaaaactcttccactgacaccaaaaccaagcaggtttcctacaaaagccctagccaaaagattaaggtgccggctggcaaaacatttcgtgttttggcgtatctgaataccggttccatcagcggtgaagcgaatctgtacgcgaacgtgggtggcatcgcctggggtgtgcttccgggttacccgaatggtggcggagtgaacatcggcgctgttttgacgaagtgccagcagaagggttggggtgatttccgcaatttccaaccgtccggtagagatgttatcgtaaaaggtcagggtacatttgagtcgaactatggtacggacttcatcctgaagatcgaagatatcaccgacagcaaactgcgcaacaacaacggttctggcaccgttgttcaagagattaaggtgccgctgattcgtaccgagatctaa

SEQ ID NO: 26 - DNA, 888 bp, synthetic construct
  source 1..888: mol_type = other DNA
atgagttcaacagatgtacaagaaaggctacgcgatctggcgcgtgaaaatgaggccggcaccttgaacgaagcgtggaataccaatttcaaaccgtccgacgaacagcaatttagctacagcccgaccgagggcatcgtttttctgactcctccgaaaaacgtcattggcgaacgtcgtatttctcagtataaggtgaacaacgcatgggcaaccttggaaggttccccgacggagatgagcggcacgccattgtacgccggtaagaacgtgctggataacagcaagggcacaagcgaccaagagctgctcaccccggaatttaactacacgtataccgagagcacctcgaacacgaccacccacggcctgaagctgggtgtgaaaacgaccgctaccatgaaattcccgattgcgcagggtagcatggaagcgtcaactgagtacaatttccaaaacagcagcaccgataccaagactaaacaggttagctacaagtccccgtcgcagaaaatcaaagttccggcaggtaaaacctttcgtgttctggcgtatctgaacaccggtagtatttccggtgaggcgaacctgtacgctaacgtgggtggcatcgcgtggggtgtattgccaggttatccgaatggtggcggagttaatattggtgctgtgcttaccaagtgccaacaaaagggttggggcgatttccgcaacttccagccgtccggccgtgacgtgatcgttaaaggccagggcacctttaccagtaattatggtactgacttcatcctgaagatcgaagatatcaccgacagcaaactgcgcaataacaacggctctgggacggttgtccaggagatcaaggtgccgttaattagaaccgagatttaa

SEQ ID NO: 27 - DNA, 888 bp, synthetic construct
  source 1..888: mol_type = other DNA
atgagttcaacagatgtacaagaaaggctacgtgatttggcgcgtgaagatgaagcgggcaccttcaatgaagcgtggaatactaattttaagccgtctgacgagcagcaatttagctattccccgactgagggcattgtctttctgaccccaccgaagaacgtgattggtgaacgtagaatcagccagtataaagtcaacaatgcgtgggcaaccctggaaggtagcccgaccgaagttagcggcaccccgctatatgttggcaaaaacgtacttgataatagcaaaggcacatccgaccaggagttgctgactccggaattcaactatacctacaccgagagcacctctaacaccacaacccacggcctgaagctgggtgtgaagaccaccgctacgatgaaatttccgatagcccaaggtagcatggaagcgagcaccgagtacaatttccaagacagctcgacggataccacgacgaagacggttagctacaaatctccgtcccagaaaatcaaagttccggcaggtaagaccttccgcgtgctcgcatacctgaataccggtagcatcagcggcgaggctaacctgtatgcgaacgttggcggcatcgcgtggggtgtgctgccgggttacccgaatggtggcggtgttaacattggtgccgtgttgaccaagtgccagcaaaagggttggggcgagttccgcaacttccagccttctggccgtgatgttattgtgaaaggtcagggtacgttcaagtccaactacggcaccgactttattctgaagatcgaagacatcaccgactccaaactgcgtaacaacaacggcagtggcaccgtcgtgcaagagatcaaagtgccattgattcgcaccgagatctaa

SEQ ID NO: 28 - DNA, 888 bp, synthetic construct
  source 1..888: mol_type = other DNA
atgagttcaacagatgtacaagaaaggctacgcgatctggcgcgtgaaaacgaagctggcaccttgaatgtggcgtggaacacgaacttcaagccgagcgatgagcagcaatttagctattctccgaccgaaggtatcgtgttcttgaccccgcctaaaaacgtcatcggtgagcgtagaatcagccagtataaggttaataatgcttgggcaaccctggaaggttccccaaccgaagttagtggtacgccactttacgcgggtaaaaacgtgctggataactcaaaaggcacgtccgaccaggagctgttaaccccggaatttaattacacctacaccgagagcacttccaatacgaccacgcacggcctgaaactgggtgttaaaaccaccgccaccatgaagtttccgattgctcagggctctatggaggccagcaccgagtataatttccaaaacagcagcaccgacactaaaacgaagcaagttagctacaagtccccgtcgcaaaaaatcaaagttccggcgggtaagacttttcgtgtattggcgtatctgaacaccggtagcatttctggcgaggcgaatctgtacgcgaacgtcggcggcattgcatggggtgttctaccgggttatccgaatggcggtggtatcaatatcggtgcagtcctgacaaagtgccaacagaagggctggggtgatttccgcaacttccagccgtccggccgtgacgtgattgttaagggtcagggcacctttaaaagcaactacggcaccgatttcatcttgaaaattgaagacattaccgacagcaagctgcgcaacaacaacggctctggcactgtggtgcaagaaatcaaggtgccgctgatccgtaccgagatttaa

SEQ ID NO: 29 - DNA, 888 bp, synthetic construct
  source 1..888: mol_type = other DNA
atgagttcaacagatgtacaagaaaggctaagagacctggcgcgtgaaaacgaggcgggaaccttgaatgaggcttggaataccaatttcaaaccgtccgatgaacagcagtttagctacagcccgaccgagggcatcgtgtttctgaccccgcctaaaaacgtcatcggcgaacgtcgtatctcgcaatataaagtgaacaacgcttgggcaaccttagaaggttccccgaccgagatgagcggcaccccgttgtacgcaggcaagaacgtgcttgataacagcaaaggcacgtccgaccaagagctgctgacgccagaatttacgtacacgtacactgagagcaccagcaacacgaccacccacggcctgaaactgggtgtgaagaccactgccaccatgaaatttccgattgcccagggctctatggaagcgagcactgagtataacttccaaaacagcagcactgacaccaagaccaagcaggttagctacaaatccccatctcagaaaattaaggtgccggcaggtaaaaccttccgcgtgctcgcgtatctgaacaccggttcgatctctggcgaggcgaatctgtacgcgaacgttggtggtattgcgtggggtgtactgccgggttatccgaatggtggcggtgttaatattggtgctgtgttgaccaagtgccagcagaagggctggggtgatttccgcaactttcaaccgagtggccgtgatgttattgttaagggtcagggtacattcacctctaattatggtacggacttcatcttgaaaatcgaagatattaccgacagcaagctgcgcaataacaacggcagcggcaccgttgttcaagaaatcaaggtcccgctgattcgtaccgagatctaa

SEQ ID NO: 30 - DNA, 888 bp, synthetic construct
  source 1..888: mol_type = other DNA
atgagttcaacagatgtacaagaaaggctacgcgacctagcgcgtgaaaatgaagctggcaccctgaatgaagcgtggaacacgaacttcaaaccgtccgacgaacaacaattttcctacagccctaccgagggtatcgtgtttctgaccccaccgaagaacgtgatcggcgaacgccgtattagccaatataaggtgaataacgcgtgggcaaccttggagggctccccgaccgaggtttctggcacaccgttgtatgctggtaaaaacgtgttggacaacagcaagggcaccagcgatcaggagctgctgacgccggaatttaactacacctataccgagagcacctcaaataccaccacccacggcctgaagctgggtgtcaaaaccactgccaccatgaagtttccgattgcgcagggcagcatggaagctagcaccgagtacaatttccaagatagcagcaccgatactacgaccaaaaccgttagctacaaatctccgtcgcaaaagatcaaagtgccggcaggcaagacgtttcgtgttttagcgtatctgaacaccggttccatttctggtgaggcgaacctgtacgccaatgttggtgggatcgcgtggggtgttctgccgggttatccgaatggtggcggtgttaacattggcgcagttttgaccaagtgccagcagaaaggctggggtgatttccgcaatttccagccgtcgggccgtgacgtgatcgttaaaggtcagggtacattcaagagcaactacggcacggacttcatcctgaaaattgaagatattaccgacagcaaactgagaaacaacaacggtagtggcacggtcgtgcaggagatcaaggtcccgctcatccgtactgagatctaa

SEQ ID NO: 31 - DNA, 888 bp, synthetic construct
  source 1..888: mol_type = other DNA
atgagttcaacagatgtacaagaaaggctacgcgacctggcccgtgaaaacgaggcgggcacgctgaacgtcgcgtggaatacgaacttcaaaccgtcagatgaacagcaatttagctacagcccgacggagggttttatctttctgaccccaccgaaaaacgtgatcggcgagcgccgtatttctcactataaagtgaacaacgcatgggcgaccttggaaggtagcccgaccgaggttagcggcacgccgctgtacgcgggtcgtaacgtgctggataattccaaaggcaccattgatcaagaaatgttgacccctgaatttaactacacctataccgaggggacctctaacaccaccacgcatggtctgaaacttggtgtcaagaccacagcgaccatgaaattcccgattgcacagggtagcatggaagcgagcaccgagtataacttccaaaacagtagcactgacaccaagacgaagcaggttagctacaagagcccgtcccagaaaatcaaagttccggcaggtaaaaccttccgcgtgttggcctatctgaataccggttccattagcggcgaagcgaatctctacgctaatgtgggcggtgtagcctggggtgtgctgccaggttatccgaatggcggcggcgtcaacattggtgctgtgttgaccaagtgccagcaaaagggctggggcgatttccgtaacttccagccgtctggtagagacgttattgttaagggtcagggcacgtttaccagcaattacggcactgacttcatcctgaagatcgaagatatcaccgactccaaactgcgtaataacaacggttcgggcacagttgttcaagagatcaaggtgccgttaattcgtaccgagatctaa

SEQ ID NO: 32 - DNA, 888 bp, synthetic construct
  source 1..888: mol_type = other DNA
atgagttcaacagatgtacaagaaaggctaagagatctggcgcgtgaagacgaagctggcacgtttaacgaagcgtggaatgcaaactttaaaccgtccgacgagccgcagtttagcaacagtccgacacagggcatcgtgatcttggcccctccgaaaaacgtgatcggtgaacgtcgtattagccaatataaagtaaataacgcgtgggcaaccctggaaggttccccaaccgaggttagcggcaccccgttgtacgcgggtcgcaacgtgcttgataattccaaaggcaccatggatcaggagctgctgaccccggaattcacgtacacctataccgagagcacctctaacaccaccactcacggcctgaagctgggtgtgaaaacgactgccaccatgaaatttccgattgcccaaggttctatggaagcgagcactgagtacaatttccaaaactcgagcaccgacaccaagacgaagcaggttagctataaatccccaagccagaaaatcaaggtgccggctgggaaaacctttcgtgtgttagcgtatctgaataccggttcgatttccggcgaggcgaacctgtacgctaacgttggtggtattgcgtggcgtgttagcccgggttatccgaatggtggtggtgtcaatattggcgcagttctcacgaagtgccagcaaaagggctggggtgatttccgcaacttccaaccgagcggccgtgatgttatcgtgaagggccagggtacgttcaagtccgactacggcactgacttcatcttgaagattgaagatatcaccgacagcaagtctcgtaacaacaacggttctggcaccgtcgttcaggagatcaaagttccgctgattcgcaccgagatctaa

SEQ ID NO: 33 - DNA, 888 bp, synthetic construct
  source 1..888: mol_type = other DNA
atgagttcaacagatgtacaagaaaggctacgcgatctggcgcgtgaaaacgaagctggcaccttaaacgtggcctggaatacgaacttcaaaccttcagacgagcaacaatttagctacagcccaacggaaggcttcatctttctgaccccgccaaaaaacgtcattggtgagcgtcgtatttcccattataaggttaacaacgcttgggcgactttggaaggttcgccgaccgaggtgagcggtactccgttgtacgcaggccgcaatgtgctggataattccaaaggtacgattgaccaggagatgctgactccggaatttaactacacctatactgagggcacctctaataccaccacgcacggtctgaagctgggtgtaaaaaccaccgcaacgatgaaattcccgatcgcgcaggggtctatggaagcgagcaccgagtataattttcaaaattccagcaccgacaccaagaccaagcaggtttcctacaagtcgccgagccagaaaatcaaagttccggcgggtaaaacctttcgtgtcttggcgtatctgaataccggtagcatttctggagaggcgaacctgtacgccaacgttggtggcgttgcatggggtgtgcttccgggttatccggatggcggtggcgttaatatcggcgctgtgttgaccaagtgccagcaaaagggttggggcgatttccgcaacttccagccgtccggtagagacgtgatcgtgaaaggtcagggcacattcaccagcaactacggcacggacttcattctcaagattgaagatatcaccgacagcaaactgcgtaataacaacggtagcggcaccgttgttcaagaaatcaaggtgccgctgattcgtaccgagatctaa

SEQ ID NO: 34 - DNA, 888 bp, synthetic construct
  source 1..888: mol_type = other DNA
atgagttcaacagatgtacaagaaaggctacgcgacctggcgcgtgaaaacgaggccggtactctgaacgaggcttggaataccaatttcaaaccgtctgatgagcaacaatttagctacagcccgactgagggtattgtgtttttgactccaccaaaaaacgtgatcggtgaacgtcgtatttcccagtataaggtaaacaacgcgtgggcaaccctggaaggttcacctacagaggttagcggcacgccgctgtacgccggtaagaacgttctggacaacagcaagggtacgatggatcaagagttgctgaccccggaatttaactacacctataccgagtccaccagcaacactacgacccacggcctgaaggtgggcgtcaaaaccaccgcaaccatgaaatttccgattgcgcagggttctatggaagcgagcaccgaatataacttccaagattccagcaccgacaccactacgaagaccgtttcctacaagagcccgtcgcaaaaaatcaaggtgccggcaggcaaaaccttcagagtcctcgcgtatttgaataccggttcgatcagcggtgaagcgaacttatatgcgaatgttgggggcattgcttggggcgtgctgccgggttacccgaatggtggcggtgtgaatattggtgctgtgttgaccaagtgccagcagaaaggctggggtgatttccgcaacttccagccgagcggccgtgacgttattgttaaaggtcagggcacgtttaagtcgaattacggcactgacttcatcctgaagatcgaagatattaccgacagcaaactgcgtaataacaacggcagtggcacagttgttcaggagatcaaagtcccgcttatccgcaccgagatctaa

SEQ ID NO: 35 - DNA, 888 bp, synthetic construct
  source 1..888: mol_type = other DNA
atgagttcaacagatgtacaagaaaggctacgcgacttggcgcgtgaaaacgaagctggcacccttaatgaagcctggaatacgaacttcaaaccgtctgatgagcagcagtttagctacagcccgaccgaaggcattgtttttctgaccccaccgaaaaacgtaatcggcgaaagacgtatttcgcaatataaagtgaacaacgcatgggcaaccttggaaggttccccgaccgaggtgtctggtactccgctgtacgtcggcaaaaatgtgctggataatagcaaaggtacaatggatcaggagctgctgaccccggaatttaactacacctataccgagtccaccagcaataccactacgcacggcttaaagctgggtgtgaaaaccacggctacgatgaaatttccgattgcgcagggctccatggaagcgtccaccgagtacaatttccaagacagcagcaccgacaccactacgaaaaccgttagctacaagtcgccgagtcaaaaaattaaggtgccggcgggtaaaacgttccgcgttctcgcgtatctgaacaccggaagcatttccggcgaggccaacctgtacgcgaacgttggtggcatcgcttggggtgtgctgccgggttatccgaatggtggtggcgttaacattggtgcagttttgaccaagtgccagcaaaagggctggggcgatttccgcaacttccagccaagcggccgtgacgtcatcgtgaaaggtcagggcacctttaagtctaactatggtacggacttcatcttgaagatcgaggacatcaccgatagcaagctgcgtaataacaacggttcaggtactgtcgttcaagagatcaaggtgcctctgattcgtaccgagatctaa

SEQ ID NO: 36 - DNA, 888 bp, synthetic construct
  source 1..888: mol_type = other DNA
atgagttcaacagatgtacaagaaaggctacgcgacctggcgcgtgaaaacgaggccggtacgttgaatgaagcgtggaatacgaactttaaaccgtctgatgaacagcaatttagctacagcccgaccgagggcattgtgtttctgactccgcctaaaaacgtcatcggcgaacgtcgtattagccaatataaagtcaataacgcgtgggcaaccttggaaggctctccgaccgaagtttctggcaccccgttgtacgttggtaaaaacgtgctggacaatagcaagggtacgatggatcaggagctgctgaccccagaatttaactacacctataccgagagcaccagcaacaccaccacccacggcctgaaggtgggtgtgaagaccaccgcaaccatgaaattcccgattgcgcagggttccatggaagcgagcacggagtataacttccaagatagcagcactgacaccactacaaaaaccgtatcctacaagagcccgtcgcaaaagatcaaggttccggcaggcaagaccttccgcgtgcttgcctacctgaacaccggtagcattagtggcgaggctaacctgtatgcgaacgttggtggtatcgcgtggggggtgctcccgggttatccgaatggtggtggcgttaatattggcgctgtgttgtccaagtgccagcaaaaaggttggggtgatttccgcaatttccagccgtcgggccgtgacgttatcgttaaaggtcagggtacgtttaagtctaattacggcactgacttcatcctgaagatcgaagatattaccgactccaaactgcgtaacaacaacggttcaggcacggtggttcaggagatcaaagtcccactgattcgtaccgagatctaa

SEQ ID NO: 37 - protein, 818 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDFR

SEQ ID NO: 38 - protein, 634 aa, synthetic construct
MDNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 39 - protein, 1177 aa, synthetic construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 40 - DNA, 2457 bp, synthetic construct
  source 1..2457: mol_type = other DNA
atggacaacaacccaaacatcaacgagtgcatcccgtacaactgcctcagcaaccctgaggtcgaggtgctcggcggtgagcgcatcgagaccggttacacccccatcgacatctccctctccctcacgcagttcctgctcagcgagttcgtgccaggcgctggcttcgtcctgggcctcgtggacatcatctggggcatctttggcccctcccagtgggacgccttcctggtgcaaatcgagcagctcatcaaccagaggatcgaggagttcgccaggaaccaggccatcagccgcctggagggcctcagcaacctctaccaaatctacgctgagagcttccgcgagtgggaggccgaccccactaacccagctctccgcgaggagatgcgcatccagttcaacgacatgaacagcgccctgaccaccgccatcccactcttcgccgtccagaactaccaagtcccgctcctgtccgtgtacgtccaggccgccaacctgcacctcagcgtgctgagggacgtcagcgtgtttggccagaggtggggcttcgacgccgccaccatcaacagccgctacaacgacctcaccaggctgatcggcaactacaccgaccacgctgtccgctggtacaacactggcctggagcgcgtctggggccctgattctagagactggattcgctacaaccagttcaggcgcgagctgaccctcaccgtcctggacattgtgtccctcttcccgaactacgactcccgcacctacccgatccgcaccgtgtcccaactgacccgcgaaatctacaccaaccccgtcctggagaacttcgacggtagcttcaggggcagcgcccagggcatcgagggctccatcaggagcccacacctgatggacatcctcaacagcatcactatctacaccgatgcccaccgcggcgagtactactggtccggccaccagatcatggcctccccggtcggcttcagcggccccgagtttacctttcctctctacggcacgatgggcaacgccgctccacaacaacgcatcgtcgctcagctgggccagggcgtctaccgcaccctgagctccaccctgtaccgcaggcccttcaacatcggtatcaacaaccagcagctgtccgtcctggatggcactgagttcgcctacggcacctcctccaacctgccctccgctgtctaccgcaagagcggcacggtggattccctggacgagatcccaccacagaacaacaatgtgccccccaggcagggtttttcccacaggctcagccacgtgtccatgttccgctccggcttcagcaactcgtccgtgagcatcatcagagctcctatgttctcctggattcatcgcagcgcggagttcaacaatatcattccgtcctcccaaatcacccaaatccccctcaccaagtccaccaacctgggcagcggcacctccgtggtgaagggcccaggcttcacgggcggcgacatcctgcgcaggacctccccgggccagatcagcaccctccgcgtcaacatcaccgctcccctgtcccagaggtaccgcgtcaggattcgctacgctagcaccaccaacctgcaattccacacctccatcgacggcaggccgatcaatcagggtaacttctccgccaccatgtccagcggcagcaacctccaatccggcagcttccgcaccgtgggtttcaccacccccttcaacttctccaacggctccagcgttttcaccctgagcgcccacgtgttcaattccggcaatgaggtgtacattgaccgcattgagttcgtgccagccgaggtcaccttcgaagccgagtacgacctggagagagcccagaaggctgtcaatgagctcttcacgtccagcaatcagatcggcctgaagaccgacgtcactgactaccacatcgaccaagtctccaacctcgtggagtgcctctccgatgagttctgcctcgacgagaagaaggagctgtccgagaaggtgaagcatgccaagcgtctcagcgacgagaggaatctcctccaggaccccaatttccgcggcatcaacaggcagctcgaccgcggctggcgcggcagcaccgacatcacgatccagggcggcgacgatgtgttcaaggagaactacgtgactctcctgggcactttcgacgagtgctaccctacctacttgtaccagaagatcgatgagtccaagctcaaggcttacactcgctaccagctccgcggctacatcgaagacagccaagacctcgagatttacctgatccgctacaacgccaagcacgagaccgtcaacgtgcccggtactggttccctctggccgctgagcgcccccagcccgatcggcaagtgtgcccaccacagccaccacttctccttggacatcgatgtgggctgcaccgacctgaacgaggactttcggtag

SEQ ID NO: 41 - DNA, 1905 bp, synthetic construct
  source 1..1905: mol_type = other DNA
atggacaactccgtcctgaactctggtcgcaccaccatctgcgacgcctacaacgtcgcggcgcatgatccattcagcttccagcacaagagcctcgacactgttcagaaggagtggacggagtggaagaagaacaaccacagcctgtacctggaccccatcgtcggcacggtggccagcttccttctcaagaaggtcggctctctcgtcgggaagcgcatcctctcggaactccgcaacctgatctttccatctggctccaccaacctcatgcaagacatcctcagggagaccgagaagtttctcaaccagcgcctcaacactgatacccttgctcgcgtcaacgctgagctgacgggtctgcaagcaaacgtggaggagttcaaccgccaagtggacaacttcctcaaccccaaccgcaatgcggtgcctctgtccatcacttcttccgtgaacaccatgcaacaactgttcctcaaccgcttgcctcagttccagatgcaaggctaccagctgctcctgctgccactctttgctcaggctgccaacctgcacctctccttcattcgtgacgtgatcctcaacgctgacgagtggggcatctctgcagccacgctgaggacctaccgcgactacctgaagaactacaccagggactactccaactattgcatcaacacctaccagtcggccttcaagggcctcaatacgaggcttcacgacatgctggagttcaggacctacatgttcctgaacgtgttcgagtacgtcagcatctggtcgctcttcaagtaccagagcctgctggtgtccagcggcgccaacctctacgccagcggctctggtccccaacaaactcagagcttcaccagccaggactggccattcctgtattcgttgttccaagtcaactccaactacgtcctcaacggcttctctggtgctcgcctctccaacaccttccccaacattgttggcctccccggctccaccacaactcatgctctgcttgctgccagagtgaactactccggcggcatctcgagcggcgacattggtgcatcgccgttcaaccagaacttcaactgctccaccttcctgccgccgctgctcaccccgttcgtgaggtcctggctcgacagcggctccgaccgcgagggcgtggccaccgtcaccaactggcaaaccgagtccttcgagaccacccttggcctccggagcggcgccttcacggcgcgtggaaattctaactacttccccgactacttcatcaggaacatctctggtgttcctctcgtcgtccgcaacgaggacctccgccgtccactgcactacaacgagatcaggaacatcgcctctccgtccgggacgcccggaggtgcaagggcgtacatggtgagcgtccataacaggaagaacaacatccacgctgtgcatgagaacggctccatgatccacctggcgcccaatgattacaccggcttcaccatctctccaatccacgccacccaagtgaacaaccagacacgcaccttcatctccgagaagttcggcaaccagggcgactccctgaggttcgagcagaacaacaccaccgccaggtacaccctgcgcggcaacggcaacagctacaacctgtacctgcgcgtcagctccattggcaactccaccatcagggtcaccatcaacgggagggtgtacacagccaccaatgtgaacacgacgaccaacaatgatggcgtcaacgacaacggcgcccgcttcagcgacatcaacattggcaacgtggtggccagcagcaactccgacgtcccgctggacatcaacgtgaccctgaactctggcacccagttcgacctcatgaacatcatgctggtgccaactaacatctcgccgctgtactga

SEQ ID NO: 42 - DNA, 3534 bp, synthetic construct
  source 1..3534: mol_type = other DNA
atggacaacaacccaaacatcaacgagtgcatcccgtacaactgcctcagcaaccctgaggtcgaggtgctcggcggtgagcgcatcgagaccggttacacccccatcgacatctccctctccctcacgcagttcctgctcagcgagttcgtgccaggcgctggcttcgtcctgggcctcgtggacatcatctggggcatctttggcccctcccagtgggacgccttcctggtgcaaatcgagcagctcatcaaccagaggatcgaggagttcgccaggaaccaggccatcagccgcctggagggcctcagcaacctctaccaaatctacgctgagagcttccgcgagtgggaggccgaccccactaacccagctctccgcgaggagatgcgcatccagttcaacgacatgaacagcgccctgaccaccgccatcccactcttcgccgtccagaactaccaagtcccgctcctgtccgtgtacgtccaggccgccaacctgcacctcagcgtgctgagggacgtcagcgtgtttggccagaggtggggcttcgacgccgccaccatcaacagccgctacaacgacctcaccaggctgatcggcaactacaccgaccacgctgtccgctggtacaacactggcctggagcgcgtctggggccctgattctagagactggattcgctacaaccagttcaggcgcgagctgaccctcaccgtcctggacattgtgtccctcttcccgaactacgactcccgcacctacccgatccgcaccgtgtcccaactgacccgcgaaatctacaccaaccccgtcctggagaacttcgacggtagcttcaggggcagcgcccagggcatcgagggctccatcaggagcccacacctgatggacatcctcaacagcatcactatctacaccgatgcccaccgcggcgagtactactggtccggccaccagatcatggcctccccggtcggcttcagcggccccgagtttacctttcctctctacggcacgatgggcaacgccgctccacaacaacgcatcgtcgctcagctgggccagggcgtctaccgcaccctgagctccaccctgtaccgcaggcccttcaacatcggtatcaacaaccagcagctgtccgtcctggatggcactgagttcgcctacggcacctcctccaacctgccctccgctgtctaccgcaagagcggcacggtggattccctggacgagatcccaccacagaacaacaatgtgccccccaggcagggtttttcccacaggctcagccacgtgtccatgttccgctccggcttcagcaactcgtccgtgagcatcatcagagctcctatgttctcttggatacaccgtagtgctgagttcaacaacatcattgcatccgacagcattactcaaatacccttggtgaaagcacatacacttcagtcaggtactactgttgtcagaggtccagggtttacaggaggagacattcttcgtcgcacaagtggaggaccctttgcttacactattgttaacatcaatggccaattgccccaaaggtatcgtgcaagaatccgctatgcctctactacaaatctcaggatctacgtgactgttgcaggtgaaaggatctttgctggtcagttcaacaagactatggataccggtgaccctttgacattccaatcttttagctacgcaactatcaacacagcttttacattcccaatgagccagagtagcttcacagtaggtgctgacactttcagctcagggaatgaagtttacatcgacaggtttgaattgattccagttactgcaaccctcgaggctgagtacaaccttgagagagcccagaaggctgtgaacgccctctttacctccaccaatcagcttggcttgaaaactaacgttactgactatcacattgaccaagtgtccaacttggtcacctaccttagcgatgagttctgcctcgacgagaagcgtgaactctccgagaaagttaaacacgccaagcgtctcagcgacgagaggaatctcttgcaagactccaacttcaaagacatcaacaggcagccagaacgtggttggggtggaagcaccgggatcaccatccaaggaggcgacgatgtgttcaaggagaactacgtcaccctctccggaactttcgacgagtgctaccctacctacttgtaccagaagatcgatgagtccaaactcaaagccttcaccaggtatcaacttagaggctacatcgaagacagccaagaccttgaaatctactcgatcaggtacaatgccaagcacgagaccgtgaatgtcccaggtactggttccctctggccactttctgcccaatctcccattgggaagtgtggagagcctaacagatgcgctccacaccttgagtggaatcctgacttggactgctcctgcagggatggcgagaagtgtgcccaccattctcatcacttctccttggacatcgatgtgggatgtactgacctgaatgaggacctcggagtctgggtcatcttcaagatcaagacccaagacggacacgcaagacttggcaaccttgagtttctcgaagagaaaccattggtcggtgaagctctcgctcgtgtgaagagagcagagaagaagtggagggacaaacgtgagaaactcgaatgggaaactaacatcgtttacaaggaggccaaagagtccgtggatgctttgttcgtgaactcccaatatgatcagttgcaagccgacaccaacatcgccatgatccacgccgcagacaaacgtgtgcacagcattcgtgaggcttacttgcctgagttgtccgtgatccctggtgtgaacgctgccatcttcgaggaacttgagggacgtatctttaccgcattctccttgtacgatgccagaaacgtcatcaagaacggtgacttcaacaatggcctcagctgctggaatgtgaaaggtcatgtggacgtggaggaacagaacaatcagcgttccgtcctggttgtgcctgagtgggaagctgaagtgtcccaagaggttagagtctgtccaggtagaggctacattctccgtgtgaccgcttacaaggagggatacggtgagggttgcgtgaccatccacgagatcgagaacaacaccgacgagcttaagttctccaactgcgtcgaggaagaaatctatcccaacaacaccgttacttgcaacgactacactgtgaatcaggaagagtacggaggtgcctacactagccgtaacagaggttacaacgaagctccttccgttcctgctgactatgcctccgtgtacgaggagaaatcctacacagatggcagacgtgagaacccttgcgagttcaacagaggttacagggactacacaccacttccagttggctatgttaccaaggagcttgagtactttcctgagaccgacaaagtgtggatcgagatcggtgaaaccgagggaaccttcatcgtggacagcgtggagcttctcttgatggaggaataa

SEQ ID NO: 43 - DNA, 888 bp, synthetic construct
  source 1..888: mol_type = other DNA
atgtcctctacagatgttcaggagcggctgcgggacctagcgcgtgaaaatgaagcaggcacgctgaacgaggcgtggaacacaaatttcaagcctagtgacgagcagcagttttcttattctcctaccgagggcatcgtcttcctcacgccgccgaagaatgtcatcggggagcgacgcatctcacagtacaaggtgaataatgcatgggctacacttgaaggctcaccaactgaagtgagcggtacgccgctttatgcagggaagaacgtcttggataattccaagggcaccatggatcaagagctattaactcccgagttcaactacacatacactgaatcaacgtccaacaccacgacccacggcctcaagctcggagtgaaaactacagccacgatgaagtttccaattgcgcagggctccatggaggcttccaccgagtacaacttccagaactcgtcaacggacaccaagacaaagcaggtttcttacaaatctccttcgcagaagataaaagttccggctggcaaaaccttccgcgtgctggcgtacctgaatactggaagcatcagcggggaagccaatttgtacgccaacgttggagggatagcctggggtgtattgcccggatatccaaatggtggcggcgtcaacattggtgctgtgctcactaaatgccagcaaaagggatggggcgactttagaaattttcaaccaagtggaagggatgtcattgtgaaagggcaaggaacattcaccagcaactatggtaccgacttcatcctgaagattgaggacattactgatagtaaacttaggaataacaacggttcggggactgtagtgcaagagatcaaggttcccctcataagaacagaaatatag

SEQ ID NO: 44 - DNA, 888 bp, synthetic construct
  source 1..888: mol_type = other DNA
atgagttccacagatgttcaagaacgcctacgcgacctcgccagggaaaatgaagcaggcaccttgaatgaggcgtggaacaccaatttcaagccatctgatgagcagcagtttagctacagcccaacggagggcatcgtcttcctcactcctcccaagaacgtcattggagagcggagaatatcacagtacaaggtgaacaatgcatgggcgacgctggaaggatcacctacagaagtaagcggtactccattatacgctggcaagaatgttttggacaacagtaaaggcacgatggaccaggagctgctaacgccggagttcaactatacctacacagaatccacttcaaatacaacaacgcacggcttgaagctcggtgtcaagaccaccgccaccatgaagttccccatcgcgcaggggtccatggaggcttctaccgagtataactttcaaaattcgtccacagatacaaaaactaagcaggtgtcgtacaaatctccgtcacaaaaaataaaagttcccgcgggaaaaacatttcgcgtgctggcatatcttaatactggaagcatttctggggaggccaacctgtacgccaatgttggtggtattgcttggggcgtgcttcctggttatccaaatggcggcgggatcaacattggcgccgtcctcaccaagtgccaacagaaggggtggggtgacttcaggaacttccagccgtcggggcgtgatgtcattgtgaagggccaaggaacattcaaatcaaactacggcactgattttattctcaagatcgaggacatcaccgactccaagctgagaaataacaacgggagtggaacggtagtgcaggaaatcaaggttcctcttatacggactgagatctag

SEQ ID NO: 45 - DNA, 888 bp, synthetic construct
  source 1..888: mol_type = other DNA
atgagctctactgatgtccaggaacgattacgcgacctcgcgcgtgagaatgaagccgggacgctgaacgaggcgtggaacacaaacttcaagccgtccgatgagcagcagttttcttacagccccactgaaggcatcgtcttcttgacaccccctaagaatgtcattggcgagcggcgcatatctcagtataaagtgtcgaacgcctgggcaacattggaggggagtccaacagaggtgtcgggcacacctctttatgccgggaagaacgtgctggataattccaaggggactatggaccaagaacttttaaccccggagttcaactacacctacactgagtctacctcaaacacgacaactcacgggctaaagcttggtgtaaaaactacggcgacaatgaagtttccaatagcgcaaggctcgatggaggcaagcaccgagtacaatttccagaattcatctaccgacactaaaaccaagcaggttagctacaagtcaccatcccagaagatcaaagtccccgccggaaaaacgtatagagttcttgcttatctgaacactggcagtatcagtggagaagctaacttgtacgctaatgttggcggcattgcatggggagttctcccgggatacccaaatggcggtggtattaacatcggtgctgtgctcacgaagtgccaacaaaagggctggggtgattttaggaattttcagccgtcaggacgggacgttattgtgaaggggcaaggtaccttcaaatccaactatggcaccgacttcattctcaagattgaagacatcacggattccaaactgagaaataacaatggaagcggtacggtagtgcaggagatcaaggtccctctgataaggacagaaatatag

SEQ ID NO: 46 - DNA, 888 bp, synthetic construct
  source 1..888: mol_type = other DNA
atgtccagtacagatgtgcaagaaagattgcgtgatctagctagagaaaatgaagctgggacgctgaatgtagcatggaacacaaatttcaagccctctgatgagcagcagttctcatatagtcctacagaaggtattgtttttttaacaccaccaaagaacgtcattggcgagcggcgaatcagccagtacaaggtgaacaacgcatgggctacccttgagggcagtcctactgaggtgagcggcacgcccctttatgcgggcaagaatgtcctggacaactccaaaggaacaatggaccaggagctcctcactccagagttcaactacacctacacagaaagcacttcaaataccacgacgcacggcctgaaacttggtgttaagacaactgccaccatgaagtttcctatcgcgcagggcagcatggaggcgtcgaccgagtacaactttcaaaattcttctactgacacaaaaacaaagcaggtctcgtacaaatcaccatcacagaaaataaaggtccccgccggcaaaaccttccgcgtgctggcatatcttaacactgggtccatctccggggaggccaacctctacgccaatgttggagggattgcttggggtgttttgcctggttacccgaatgggggcggcatcaacattggtgccgtactcaccaagtgccaacaaaagggatggggcgacttccggaacttccagccgtcggggagggatgttattgtgaagggccaaggaacgtttaagagcaactatggtactgatttcatcctcaagatagaggacatcacggactccaaattgcgcaataataatggatctggaaccgtcgtgcaggaaatcaaggtgccgctgataaggacggagatatag

SEQ ID NO: 47 - DNA, 888 bp, synthetic construct
  source 1..888: mol_type = other DNA
atgtccagtactgatgtgcaagagaggctccgcgacctagcccgggaaaatgaagctggcacgctcaacgtagcttggaacacaaatttcaagccgtccgacgagcagcagttttcgtactcccctactgaaggaattgtgtttcttacgccgcccaagaacgttattggtgaaagaaggatttctcagtacaaggtcaataatgcgtgggcgacgctggaggggtctccaacagaagtttcaggaactcccctctatgcaggcaagaatgtgctcgacaacagcaagggcacaatggatcaggagcttttgacacctgagttcaactacacctacacggagtctacatcaaatacaactacgcacgggctgaagctcggcgttaagaccaccgccaccatgaagttccccatcgcgcagggctccatggaggcgtcgactgagtacaacttccagaactccagcactgacaccaaaactaagcaggtgtcgtataagagcccatctcaaaagatcaaggttcctgctgggaagacatatagagtcctagcatatctgaacaccggcagcataagcggggaggccaacctgtacgccaatgttggtggtattgcatggggcgtccttccgggctacccaaatggaggcggcatcaacatcggcgccgtcttaacaaaatgccaacagaaggggtggggtgattttcgaaattttcaaccaagtggacgtgatgtcatagtaaaaggacaaggtaccttcaaatcaaattatgggacggacttcatcttgaaaattgaagacatcacagattcaaaattgcgcaacaacaatggtagtggaacggtggtgcaggaaataaaagtgccgctgattcggaccgagatctag

SEQ ID NO: 48 - DNA, 888 bp, synthetic construct
  source 1..888: mol_type = other DNA
atgtcatctactgatgttcaggagaggctaagggatcttgcaagagaaaatgaagctggtacgctcaatgtggcgtggaacacaaatttcaagccatctgatgagcagcagttctcctattctccaacagaaggtattgtattccttacgccgcccaagaatgtcataggtgaacgtcggatttcacagtacaaggtcaacaatgcatgggcgacgctggaaggaagtcctaccgaggtgtccggaactcctctgtacgccggcaagaacgtccttgacaacagcaagggtacaagtgatcaagaacttttaacacctgagttcaactacacctatacagagtcgacgagcaacaccaccacgcacggcctcaagctgggggtgaaaactaccgccaccatgaagtttccaattgctcaaggatccatggaggcctcgacggagtacaacttccagaattcatcgaccgacacaaaaacaaagcaggttagctacaaaagcccgtctcagaagatcaaagttcctgctgggaagacttatagggtgctggcgtacctcaacactggcagcatatccggcgaggcgaatttgtatgcaaatgttggaggcatcgcttggggtgtgctccccggctacccaaatggcggcgggatcaacatcggcgccgtcctcaccaagtgccagcaaaagggatggggcgacttccgcaactttcagccgtcggggcgcgacgtcatcgtgaaggggcaaggcacctttaaatcaaattatggaacagattttatactgaaaattgaggacatcacagactccaaattgcggaataacaacggtagtggtactgttgtacaagagatcaaggtgcccttgattagaactgaaatatag

SEQ ID NO: 49 - DNA, 888 bp, synthetic construct
  source 1..888: mol_type = other DNA
atgtccagcacggatgtccaagaacgtctgcgcgacttggcacgcgagaatgaggcggggaccctaaacgaggcctggcacacaaatttcaagccctccgacgaacaacagttctcctatagcccgacggaaggcatcgtgttcctcactccaccaaagaacgtaataggcgagaggagaatttctcaatataaggtgaacaatgcatgggctacactggagggcagcccaaccgaggtctcaggcaccccgctctacgcggggaaaaatgtgctcgacaactctaaaggaacaatggaccaggagcttctgacgccggagttcaactacacgtacacagaaagtactagtaacacgattacccatggattgaagctcggagtgaaaactactgctactatgaagttccccatcgcccaaggctccatggaggcctctacggagtacaacttccagaattcgtctaccgacaccaagacaaagcaggtctcgtacaagtcaccgagccagaagattaaagttcctgcaggcaagacatttagggtcctcgcgtacctgaacactggtagcatttctggtgaagctaacctttatgctaatgttggagggatcgcgtggggcgttcttcctggttatcctaatggtggcggcgtgaacattggagccgttttgacaaaatgccagcagaaagggtggggtgactttaggaattttcagcccagtgggcgggatgtaattgtcaagggccagggtacctttgagtcgaattatggaacagatttcatattaaagatcgaagatatcactgattcaaagctgcggaataacaacgggtcaggaaccgtggtgcaagagatcaaagttcctcttatcagaacggaaatatag

SEQ ID NO: 50 - DNA, 888 bp, synthetic construct
  source 1..888: mol_type = other DNA
atgagctcaacagatgtgcaggagcggctgcgcgatctggcgcgtgaaaatgaggctggaaccttgaatgaagcatggaacaccaacttcaagccatcggacgagcagcagttttcatacagcccgacggaaggtattgttttcctaacgccgcccaagaacgtcattggagaacggcgcatctctcaatacaaggtgaacaacgcctgggcaactcttgagggctcgcctactgagatgtcagggactccgctctatgctgggaagaatgtgctggacaactcaaaaggaacttctgatcaagaattacttacaccagagttcaactacacctatacggaaagtacgtccaacacaaccacacacggcctcaagttaggtgtgaagacgacggcgaccatgaagttccccatagcacaggggagcatggaggcgtcgacagagtacaacttccagaatagttcgactgatacaaaaacaaagcaggtgagctacaaatctcctagtcaaaagataaaagttcctgctggcaaaacttttagagtactcgcctaccttaatactggatccatctccggcgaggccaacttgtatgctaatgtcggcgggatcgcgtggggcgtgctccccgggtaccctaatggtggtggcgtcaacatcggcgccgtcctcaccaagtgccagcagaagggctggggtgacttcaggaattttcagccatctggaagagacgttattgtcaaaggtcaaggaaccttcacttccaattatggcaccgattttatattgaaaattgaggacatcacggactccaagctgaggaacaataatggcagcgggactgtagttcaagagatcaaggttccgctgatccgaacagaaatttag

SEQ ID NO: 51 - DNA, 888 bp, synthetic construct
  source 1..888: mol_type = other DNA
atgtcctccaccgacgtgcaagagcgcctccgggaccttgccagagaggatgaggctggcacctttaatgaagcatggaacacaaatttcaaaccaagcgacgagcaacaattttcttattctccgacggagggaatcgtcttcctcacacctccaaaaaacgtgattggcgaacgacggatatctcagtacaaggttaataatgcctgggccacgctggaaggctctcctaccgaggtttcagggactccgctatatgttggtaagaacgtcctggacaactcgaaggggacctcggatcaggagctcctaacaccggagttcaactacacgtatactgaaagtaccagtaacacgaccacccacgggcttaagttgggcgtgaaaactactgcaacgatgaagttccccatcgcgcagggaagcatggaggctagcactgagtacaacttccaggatagctccacagacacaaccactaaaaccgtcagctacaaatcaccctcacaaaaaatcaaggtgcccgcgggcaagactttccgcgtattggcgtacctgaatacaggaagtatttcgggagaggccaacctctatgcaaatgtgggcgggatagcttggggtgttcttcctggatacccaaatggagggggtgtaaacatcggcgcggtgctgaccaagtgccagcagaagggctggggcgagtttaggaattttcagccatccgggcgtgatgtaattgtcaagggtcaaggcacatttaagtcaaattatggtacggacttcattttgaagatcgaagatataactgattccaagttaaggaacaacaatggctccgggacggtcgtgcaggaaattaaagttccgctgatcagaacagaaatatag

SEQ ID NO: 52 - DNA, 888 bp, synthetic construct
  source 1..888: mol_type = other DNA
atgtcatctactgatgttcaagaacgcctccgtgatttagcgcgcgagaatgaagctgggactttgaatgtagcttggaacactaatttcaagccttcagatgagcagcaattttcttattctccaacagaagggatcgtcttcctcaccccgcccaagaacgtcatcggcgagaggaggatatcacagtacaaagtcaacaacgcgtgggcgacgctggaaggaagtcccactgaggtgtccggcacaccactatatgcagggaaaaatgttctggacaattccaaaggaacatctgatcaagagctgctcacgccggagttcaactacacatacactgagagcactagcaacaccacgacgcacggcctcaagctaggcgtcaagaccacagccaccatgaaatttccaattgctcagggctccatggaggcatcgacggagtacaacttccagaacagctcgactgacaccaagacgaagcaggtttcttacaagagtccttcccaaaagataaaagtgccggcgggtaagacatttcgggtgctggcctacctcaacaccgggagcatctcgggggaggccaatctttatgccaacgtcggcggaattgcatggggcgtgttgccgggctacccaaatggaggtggcatcaacattggtgctgtattgacaaaatgccagcagaaggggtggggagatttcagaaattttcagccctcaggtagagacgtgattgtgaagggccaaggaaccttcaaatcaaattatggtaccgacttcatcctgaagattgaagacataacagacagcaaacttcggaacaataatggcagtggtacggttgtgcaggagatcaaggttcctcttatccgaactgaaatttag

SEQ ID NO: 53 - DNA, 888 bp, synthetic construct
  source 1..888: mol_type = other DNA
atgagttctactgatgtgcaggaaaggttgcgtgacttagctagagaaaatgaagccggcaccctaaatgaggcatggaatactaatttcaagccctctgatgagcaacagttctcttacagcccaactgaggggatcgtcttcttgactcctccaaagaacgtcataggagaacgacgcatttcacagtacaaggttaataatgcttgggcgacgctggaaggtagccccaccgagatgtcgggtacacctctttatgctgggaaaaatgttctggacaactcgaaaggaacaagtgatcaggagctcctgacgccggagttcacatatacgtacactgaatcaacctcaaacacaacaacccacggcctgaaacttggcgtcaaaacaacagccaccatgaagtttcctatagcacagggctccatggaggcgagcaccgagtacaacttccagaattcttccactgacaccaaaactaagcaagtttcttataagagcccttcacaaaagatcaaggtcccggcgggcaagacatttagagttcttgcatatctcaacaccgggtccataagcggggaggctaacctgtacgccaacgtgggcggcatcgcgtggggagtattgcccggctacccaaatggtggtggtgtgaacatcggcgccgtcttaacgaagtgccagcagaagggctggggcgactttaggaactttcagccatcggggagggacgttatagtgaaggggcaaggtacgttcacttccaattatggaaccgacttcatcctcaaaattgaagatattactgattccaagctccgcaacaacaatggaagtggtacggtggtgcaagagattaaagtaccgctcatccggacggagatttag

SEQ ID NO: 54 - DNA, 888 bp, synthetic construct
  source 1..888: mol_type = other DNA
atgtcgtccactgatgttcaagagaggcttcgggacctggcgcgggagaacgaggcgggcaccctcaacgaagcttggaacactaattttaagccttccgatgagcagcaattctcatattcaccgaccgaaggcatcgtgttcttgactccaccaaagaatgttattggggagaggcgaatctcgcagtacaaggtgaacaacgcgtgggcgacgctggaaggttctccgacggaggtcagtggaacccccctttacgccggtaagaacgttcttgacaattcaaaaggaacatcagatcaggaactgcttacgcccgagttcaactacacttatactgaaagcacaagcaacacgacaacacacggactgaagctgggcgtgaagacgaccgctaccatgaagtttccaattgctcaaggatctatggaggcaagtacagagtacaactttcaggacagctccaccgatacaaccactaaaacagtctcctataaatctccctcgcagaagatcaaggtcccggcagggaaaacatttcgtgtgctagcctacttgaacaccgggagcatatctggtgaagcaaatttgtatgccaatgtaggtggtatagcttggggcgtattacctggctacccgaatggcggcggggtcaacataggcgccgtgctcacaaaatgccagcagaaaggctggggtgatttcagaaatttccaaccttctgggagagacgtgatcgtcaagggccagggcactttcaagagcaattatggaactgacttcattctgaagattgaggacatcaccgatagtaagctccgcaacaataatgggtcagggacggtggttcaagaaataaaagttccactcatccgcactgagatttag

SEQ ID NO: 55 - DNA, 888 bp, synthetic construct
  source 1..888: mol_type = other DNA
atgtcttctactgatgttcaagagcgcctccgagacttggctagggaaaatgaagccggcactcttaacgtggcgtggaacacaaatttcaagcctagtgatgagcagcagttctcgtatagcccgactgagggcttcatttttttaacgccgccgaagaatgtcattggagagcgtcggatctcccactataaagtcaataacgcctgggcgacgctggaagggtcccccacagaggtgtcaggtacccctctgtacgccggcaggaatgttttggacaacagcaagggcaccatcgaccaggagatgctgacgccagagttcaactacacctacacagaaggaacttcaaacacaacgacccatgggctgaaactcggggtgaagactacagccaccatgaagtttcctattgcgcagggcagcatggaggcatcaacggagtacaacttccagaactcatctacagatacaaaaactaagcaagtaagctacaagagtccttctcagaagatcaaggtgcccgcgggcaagacctttcgggtgctggcatacctaaacacaggatccatcagtggtgaagctaatctctatgctaacgtcggtggggttgcttggggcgtccttcccggctacccaaatggaggcggggtaaatattggcgcagtgctcaccaaatgccagcaaaaagggtggggagacttcagaaactttcagccgagcggaagagatgtcatcgtcaaggggcaaggaacgttcacttcgaattatggtacggattttatattgaagattgaggacataaccgactcgaaacttcgcaacaataatggttccggcacagtggttcaagaaatcaaagttccactcatcaggactgaaatatag

SEQ ID NO: 56 - DNA, 888 bp, synthetic construct
  source 1..888: mol_type = other DNA
atgagctcaactgacgtgcaggaaaggctgcgggacctcgccagggaggacgaggccggcacgtttaatgaagcgtggaacgcaaatttcaaaccttctgatgagccgcagttctccaattcaccaacacaaggtatagtgatacttgctcccccaaagaacgtcattggtgaaagaagaatatcccagtacaaagtcaataatgcctgggcgacgctggaggggtctccgacggaggtctcgggcactccactatacgcggggcgcaacgtgctggacaattctaaaggcaccatggaccaagagcttttgacgccggagttcacctacacatatactgagagcacctctaacacaacgacgcacggcttaaaactgggagtgaagacaactgcaacaatgaagtttcctattgctcagggcagcatggaggcaagtactgaatataattttcaaaacagctcgacagatacaaaaacaaagcaggttagctacaagtccccatcgcagaagatcaaggtgcctgctggcaagaccttccgcgtattggcctacctcaacaccggatctattagtggagaagccaacctttatgctaatgttggtggaattgcatggcgtgtttcaccgggctaccccaacggaggtggcgtgaatatcggcgcggtcctaaccaagtgccagcagaaagggtggggcgacttccggaacttccagccctcggggcgcgatgtaatagtcaaggggcaagggacgttcaagagtgattatggaactgattttatcctcaagattgaagacatcaccgattcaaagtccaggaacaacaatggatcaggtactgttgtgcaagaaattaaagttcctctcatccgaaccgagatctag

SEQ ID NO: 57 - DNA, 888 bp, synthetic construct
  source 1..888: mol_type = other DNA
atgagctctaccgacgtgcaagagagactccgtgacctggcccgggaaaatgaagctggcactcttaatgtagcgtggaacacgaacttcaagccttcagatgaacagcagttcagttacagccctactgaagggttcatctttctgacgccccccaagaacgtaatcggcgagcgcaggattagccactacaaggtgaataacgcctgggcgacattggagggatcaccaacggaagtttcgggtacgcccctgtatgctgggaggaatgttctcgacaatagcaaggggaccattgatcaagagatgttaaccccggagtttaactacacctacactgagggcacttcaaatacaacaactcatggtctcaaattgggtgtcaagacaacagccaccatgaagttcccaatagcccagggctccatggaggcgagcacagagtacaattttcagaattcttccactgacacgaagaccaagcaagtctcctacaaatctccgtcccagaagatcaaagtcccggctggaaaaacatttcgagtcctcgcgtaccttaacacggggtcgatctcaggtgaagcaaatctatatgcaaacgttgggggcgtggcatggggtgttctacctggctatccagatggtggcggagtgaacataggcgccgtccttactaaatgccagcaaaaaggatggggcgacttcaggaacttccagccttctgggcgggacgtcattgtgaagggccaaggaacgttcaccagtaactatggaactgattttatattgaagattgaggatattacagatagtaaactgcgcaacaacaatggttcgggcaccgtggtgcaggagatcaaggttccgctgatcagaacagaaatatag

SEQ ID NO: 58 - DNA, 888 bp, synthetic construct
  source 1..888: mol_type = other DNA
atgagcagtacagatgttcaggagcgcttgagggatcttgcgcgggagaacgaggctggaaccttgaatgaagcatggaacaccaactttaagccatcagatgagcagcagttctcctattctcccacggaaggaattgtcttcctcacgccgcccaagaacgtcatcggcgagagacggatatctcaatataaggtgaataatgcttgggcgacgttggaaggctcacctacagaggtctctggaacaccgctttatgccgggaaaaatgtactagacaactctaaaggaacaatggatcaagaactgctgactcctgagttcaactatacctacacagaatcaacatccaacactacgacgcacggtctcaaagtcggcgtgaagacgaccgcgaccatgaagtttcccatagcacaggggagcatggaggcctccactgagtacaatttccaggactcgtcaactgacacgactacaaagactgtatcctacaagagtccatcccagaagatcaaggttcctgctggcaaaacatttcgcgtgcttgcatacctcaatactggaagcatttctggggaagccaacctgtatgctaatgttggtggtatcgcgtggggtgttctgccgggctacccaaatggggggggcgttaacattggcgccgtgttaaccaagtgccagcaaaaaggctggggcgactttagaaatttccaaccatcgggccgagatgtgatcgtcaaggggcagggtaccttcaagagcaactacggcactgatttcattctcaaaattgaggacatcaccgacagcaagctgcgtaataacaatggttcgggaactgtggtgcaagagattaaagtcccgctcatcaggaccgaaatatag

SEQ ID NO: 59 - DNA, 888 bp, synthetic construct
  source 1..888: mol_type = other DNA
atgtcatcaactgatgtacaagagaggctgcgagacctggctcgggaaaacgaggctgggaccctcaatgaggcctggaacaccaattttaaaccgagcgatgagcagcagttctcgtattcccccactgaagggatagtgttcctgactcctccgaagaatgtaatcggcgagaggagaatctcacaatacaaggtcaataatgcgtgggctacactagaaggttctccaacggaagtcagcggcaccccattgtacgttgggaagaacgtgttagacaattccaaaggcacaatggaccaagaacttcttacgcctgagttcaactacacgtacactgaatctacgtcaaatactacgacgcacgggttgaagcttggggtgaaaaccactgcgaccatgaagtttcctatagcacagggctcgatggaggcctctaccgagtacaacttccaggattcctccacggataccactacaaaaacagttagctacaagagtcctagtcagaagataaaagtccccgccggaaaaacatttcgggtccttgcatatctcaacaccggtagcattagtggtgaagccaatctctacgcaaatgtcggtggaatagcgtggggcgttctccccggatatccaaacggtggaggcgtgaacatcggcgcggtgctgacaaagtgccagcaaaaagggtggggcgattttcgcaacttccagccgtccggccgcgacgtgattgtgaagggacaaggaacattcaagtcgaactatggtaccgatttcattttaaagatcgaggacattactgacagcaagctgagaaataacaacggctctgggactgttgttcaggagatcaaggtaccattgattcgtacagagatctag

SEQ ID NO: 60 - DNA, 888 bp, synthetic construct
  source 1..888: mol_type = other DNA
atgtcatctaccgatgtgcaagaaaggctccgcgacctcgcgagggagaacgaggccggcacgctcaatgaagcgtggaacacaaatttcaagccttctgatgagcagcagttctcgtacagcccaactgaaggaatagtgtttctgacgccgccaaaaaatgtcatcggcgagcggcgcatatcacagtacaaagtcaataatgcttgggccacccttgaaggcagccccaccgaggtgtcaggaactcctctgtatgtaggcaagaatgttcttgacaactccaagggtaccatggaccaggagttgttgacgcctgagttcaactacacctatacagaaagtacaagcaatacaacgacgcacggcctcaaggtgggggtgaagacaactgctacaatgaagttccccatcgcgcaaggaagcatggaggcatcaacagagtacaacttccaggactcgtccactgatacaaccaccaaaaccgtctcctacaagagcccgtcccagaagatcaaagttccggcggggaagacttttcgtgtgctcgcctacctgaacactggaagtatctctggagaagcaaacctatacgccaacgttggcgggattgcatggggtgtactgccggggtatccaaatggtggcggcgtcaacattggtgctgttttatccaagtgccagcaaaaaggctggggcgactttagaaattttcagcccagtggtcgggatgttattgtcaagggacaaggcaccttcaaatctaactatggtactgacttcatcctgaagattgaagatattacagattcaaaactaaggaacaacaatggatcggggacggtggtgcaagagatcaaggttcctcttataagaactgagatatag

(19) United States
(12) Patent Application Publication
(10) Pub. No.: US 2023/0180767 A1
(43) Pub. Date: Jun. 15, 2023

(54) Use of insecticidal protein

(71) Applicant: BEIJING DABEINONG BIOTECHNOLOGY CO., LTD., Beijing (CN)

(72) Inventors: Tengyu CHANG, Beijing (CN); Qinyang WANG, Beijing (CN); Aihong ZHANG, Beijing (CN); Qing TAO, Beijing (CN)

(21) Appl. No.: 18/062,607

(22) Filed: Dec. 7, 2022

(30) Foreign Application Priority Data
Dec. 13, 2021 (CN) 202111515716.1

(51) Int. Cl. A01N 61/00; A01P 7/04; C12N 15/82

(52) U.S. Cl. CPC A01N 61/00; A01P 7/04; C12N 15/8286

(57) ABSTRACT

Related is a use of an insecticidal protein. The insecticidal protein may be used to control Ostrinia furnacalis (Hubern). A method for controlling Ostrinia furnacalis (Hubern) includes: allowing the Ostrinia furnacalis (Hubern) to be at least in contact with an ACe1 protein. In the present application, the ACe1 protein that can kill the Ostrinia furnacalis (Hubern) is produced in bacteria and/or a plant body to control the Ostrinia furnacalis (Hubern).

### CROSS-REFERENCE TO RELATED APPLICATION

The present application is based on and claims priority to Chinese application with a CN application number of 202111515716.1 and an application date of Dec. 13, 2021, the disclosure of which is hereby incorporated by reference again in its entirety.

### SEQUENCE LISTING

The instant application contains a Sequence Listing which has submitted electronically in ASCII format and is hereby incorporated by reference in its entirety. Said ASCII copy is named PN192853 SEQ LIST.xml and is 100,287 bytes in size. The sequence listing contains 60 sequences, which is identical in substance to the sequences disclosed in the CN application and includes no new matter.

### TECHNICAL FIELD

The present application relates to a use of an insecticidal protein, and in particular, to a use of an ACe1 protein for controlling damage of Ostrinia furnacalis (Hubern) to a plant by expressing in the plant.

### BACKGROUND

Ostrinia furnacalis (Hubern) is a holometabolous insect belonging to Lepidoptera Pyralidae. The pest mainly damages corn, sorghum, millet, and can also damage crops such as cotton, sugar cane, hemp, sunflowers, rice, sugar beet, and beans, which belongs to a worldwide pest. Damage occurs in all corn belts in China, so that the pest is the number one pest in corn production. In normal years, the damage of spring corn decreases by 7-10%, and in large occurrence years, production is reduced by more than 30%. Due to short growth seasons of summer corn, damage caused by corn borer is larger. Corn is one of the largest food crops in China, with an annual planting area of 600 million mu. Calculated based on the yield of 400 kilograms per mu, the annual yield reduction due to the corn borer damage is 24 billion kilograms, equivalent to about 30 billion yuan.

The Ostrinia furnacalis (Hubern) goes through 4 stages in its life: egg, larva, pupa and adult. Generally, 1-7 generations may occur in a year (depending on the latitude). The larva stage is the period that the pest damages the corn. In Huang-huai and Northeast regions where the corn is grown, there are at least two generations of larvae a year that can damage the corn. In the northern spring corn area, the first generation of larvae mainly damage the corn seedling stage. Once hatched, the larvae dive into the heart leafage to eat the heart leaf or feed on the mesophyll of the tender leaf between the leaves, resulting in pinholes or window paper-like leaves. If the larvae over 3 years old bore into the unexpanded heart leaves, the larvae feed on the heart leaves, resulting in rows of holes. When the larvae feed on the heart leaves and develop to around the 3rd instar, the corn enters the trumpet period, and tender tassels are more attractive to the corn borer than the heart leaves, so that the larvae moves to feed on ears. After tasseling, the larvae are taken out from the heart leaves, and in this case, all larvae begin to transfer downwards, except for a few mature larvae that can pupate on the tassels. Since the larvae are basically 4-5 instars at this case, the larvae begin to exert “boring” characteristics. The ears and upper and lower nodes of the ears are the most vulnerable parts. In this case, the ears of the corn have begun to develop. If the nearby stem nodes are eaten, the normal development of the ears may be obviously affected or even stops. In addition, since the corn borer bores the stems in the middle and lower parts of the plant, it is easy to cause the plant to downturn in the wind, making the loss even greater. The second generation of the pest occurs in the silking stage of the corn. Most of the newly hatched larvae hide in the bases of the filaments at the tops of the ears, feed on the filaments and then feed on the tender grains, and a few goes to the leaf axils to feed on the accumulated pollen or leaf axil tissue. After the larvae develop to 3 instars, the larvae begin to bore, or directly bore into the cobs from the ear heads or continue to feed on the grains that are filling, or transfer downward from the tops of the ears, and then bore into the ears, the ear stalks or the stems again. In this case, the ears of the corn have fully developed to proper sizes and have entered the milk-ripe stage. As a result, the damage at the ear stage does not affect the sizes of the ears, but affects the normal grain filling of the corn, thereby reducing thousand seed weight. If the ear stalks are bored, the ears are broken and fallen off. In addition to directly causing yield loss, the larvae feeding on grains often causes the infection of corn ears and stem rot, which worsens yield losses and quality losses.

In China, the control of the Ostrinia furnacalis (Hubern) began in the 1950s. Main control methods usually used are agricultural control, chemical control, physical control, sex pheromone control and biological control.

The agricultural control is to comprehensively coordinate and manage an entire farmland ecosystem from multi-factors, and regulate crops, pests, and environmental factors so as to create a farmland ecological environment that facilitates crop growth but not facilitates the occurrence of the Ostrinia furnacalis (Hubern). For example, measures of treating the overwintering host of the corn borer, reforming the farming system, planting borer-resistant varieties, setting up trap fields and intercropping can reduce the damage of the corn borer. Since the agricultural control must obey the requirements of crop layout and yield increase, the application of the agricultural control has certain limitations and cannot be used as an emergency measure, which appears powerless when the borer pest outbreaks.

The chemical control is pesticide control, which uses chemical pesticides to kill pests and is an important part of the comprehensive management of the Ostrinia furnacalis (Hubern). The chemical control has the characteristics of rapidity, convenience, simplicity and high economic benefits, and is an essential emergency measure, especially in the case of a large occurrence, which may eliminate the pest before the pest causes damage. For example, the granule control method with the largest promotion area has the most stable and reliable effect currently, but the tool for efficient granule application is still far from reaching a standard, which seriously affects the achievement of the effect of the granule. In addition, there are pesticide control methods such as poisonous soil spreading, liquid medicine spraying, dichlorvos stacking, and a method of using dichlorvos to stack and fumigate the overwintering generation of adults in straw stacks. However, the chemical control also has its limitations. For example, the improper use may often lead to adverse consequences such as the phytotoxicity of crops, the drug resistance of pests, the killing of natural enemies, and the pollution of the environment, so the farmland ecosystem is destroyed and pesticide residues pose a threat to the safety of humans and animals.

Physical control is mainly based on the response of pests to various physical factors in environmental conditions, using various physical factors such as light, electricity, color, temperature and humidity, as well as mechanical devices for trapping, radiation sterility and other methods to control pests. Currently, the most widely used is high-pressure mercury lamp trapping, which takes advantage of the characteristics that the overwintering larvae are concentrated in the corn stalk stacks in the villages. During the eclosion period of the overwintering generation, high-pressure mercury lamps are set up in the villages on a large scale to trap and kill the adults of the corn borer, and the control effect is obvious. The high-pressure mercury lamps must be used in villages that ensure continuous power supply at night, and the operation is difficult.

The sex pheromone control is to use synthetic sex pheromone of the corn borer to directly trap and kill the male corn borer and interfere with the mating of the corn borer, so that the fertilization rate of females in the fields can be reduced, thereby alleviating borer pest.

The trapping method is to use the synthetic sex pheromone of the corn borer in mating habitats of the corn borer such as well-growing wheat or vegetable fields, to trap and kill the male corn borer. The disadvantage of this method is that the management of basin traps requires a lot of labor, and the method must be used in a large area to be effective.

The mating disruption method is to, when 10% of the overwintering corn borer adults have emerged, use 4500-6000 sex pheromone dispensers per hectare to hang on the crops in the mating sites of the corn borer, so as to interfere with the mating of the moths. Using the sex pheromone to control the first generation of corn borer is simple in method, desirable in effect, pollution-free to environment, and no harm to natural enemies. The downside is the delivery cost of trapping cores.

The biological control is the use of some beneficial organisms or biological metabolites to control the number of pest populations in order to achieve a purpose of reducing or eliminating the pests. Its characteristics are that it is safe for the humans and animals, low in the pollution of the environment, and may achieve a long-term control purpose of certain pests. The most common biological control is Trichogramma and Beauveria bassiana. However, the Trichogramma control largely affects by climate factors, so that the effect is often unstable. In addition, the same cost is required regardless of the severity of the borer occurrence.

In order to solve the limitations of the agricultural control, the chemical control, physical control and the biological control in practical applications, it is found from researches by scientists that some insect-resistant transgenic plants may be obtained by transferring insect-resistant genes encoding insecticidal proteins into plants so as to control plant pests.

ACe1 is a new class of insecticidal proteins, which is completely different from the traditional Bt protein. By analyzing a protein secondary structure, the protein is speculated to belong to a β-pore forming protein. The mechanism of action of such proteins is generally enzymatic cleavage activation, binding with receptors, formation of oligomers, and pore-forming on membrane surfaces. The enzymatic cleavage activation in insect gut, receptor binding on the insect gut and a physicochemical environment in the insect gut determine whether the protein can form in cell membranes of the insect gut. After such type of protein is secreted by the bacteria, it needs to be digested in a target body to form an active protein. The enzyme cleavage process is mainly performed at an amino-terminal or carboxyl-terminal of the protein, to turn the protein into an active fragment. The active fragment binds to a receptor on an epithelial cell membrane of the insect gut to form oligomer, and inserts into an intestinal membrane, so that a perforated lesion appears on the cell membrane, and the osmotic pressure change and pH balance and the like inside and outside the cell membrane are destroyed, and the digestion process of the insects is disrupted, finally resulting in death of the insects.

The ACe1 protein has been reported to have inhibitory activity against Coleoptera corn rootworms. However, there is no report on the control of plant damage by the Ostrinia furnacalis (Hubern) by producing transgenic plants expressing the ACe1 protein so far.

### SUMMARY

The present application is intended to provide a use of an insecticidal protein, and for the first time provide a method for controlling Ostrinia furnacalis (Hubern) by producing a transgenic plant expressing an ACe1 protein, to effectively overcome technical defects in agricultural control, chemical control, physical control and biological control in the prior art.

In order to achieve the above objective, the present application provides a method for controlling Ostrinia furnacalis (Hubern), including allowing the Ostrinia furnacalis (Hubern) to be at least in contact with an ACe1 protein.

Further, the ACe1 protein is present in a host cell that produces at least the ACe1 protein, and the Ostrinia furnacalis (Hubern) is in contact with at least the ACe1 protein by ingesting the host cell.

Further, the ACe1 protein is present in bacteria or a transgenic plant that produces at least the ACe1 protein, the Ostrinia furnacalis (Hubern) is in contact with at least the ACe1 protein by ingesting the bacterium or a tissue of the transgenic plant, and after contacting, the growth of the Ostrinia furnacalis (Hubern) is inhibited and/or death is caused, so as to achieve the control of the damage of the Ostrinia furnacalis (Hubern) to plants.

The transgenic plant may be in any growth stages.

The tissue of the transgenic plant is a root, a leaf, a stem, a tassel, an ear, an anther, or a filament.

The control of the damage of the Ostrinia furnacalis (Hubern) to the plants does not vary with planting location and/or planting time.

The plant is corn and sorghum.

A step before the contacting step is to plant a plant containing polynucleotide encoding the ACe1 protein.

On the basis of the above technical solution, the ACe1 protein is an ACe1_3 protein, an ACe1_4 protein, an ACe1_5 protein, an ACe1_6 protein, an ACe1_8 protein, an ACe1_9 protein, ACe1_10 protein, an ACe1_11 protein, an ACe1_12 protein, an ACe1_13 protein, an ACe1_14 protein, an ACe1_15 protein, an ACe1_16 protein, an ACe1_17 protein, an ACe1_18 protein, an ACe1_19 protein, an ACe1_20 protein, or an ACe1_21 protein.

Preferably, the ACe1 protein has an amino acid sequence shown in any one of SEQ ID NO:1 to SEQ ID NO:18.

On the basis of the above technical solution, the plant further includes at least one second nucleotide different from nucleotide encoding the ACe1 protein.

Further, the second nucleotide encodes a Cry-like insecticidal protein, a Vip-like insecticidal protein, a protease inhibitor, lectin, α-amylase, or a peroxidase.

Preferably, the second nucleotide encodes a Cry1Ab protein, a Cry2Ab protein, or a Cry1A.105 protein.

Further, the Cry1Ab protein, the Cry2Ab protein, or the Cry1A.105 protein has an amino acid sequence shown in SEQ ID NO:37, SEQ ID NO:38, or SEQ ID NO:39, respectively. The second nucleotide has a nucleotide sequence shown in SEQ ID NO:40, SEQ ID NO:41, or SEQ ID NO:42.

Optionally, the second nucleotide is a dsRNA that inhibits an important gene in a target insect pest.

In order to achieve the above objective, the present application further provides a use of an ACe1 protein for controlling Ostrinia furnacalis (Hubern).

In order to achieve the above objective, the present application further provides a method for producing a plant for controlling Ostrinia furnacalis (Hubern), including introducing a polynucleotide sequence encoding an ACe1 protein into a genome of the plant.

In order to achieve the above objective, the present application further provides a method for producing a plant seed for controlling Ostrinia furnacalis (Hubern), including hybridizing a first plant obtained by the method with a second plant, so as to produce a seed containing a polynucleotide sequence encoding an ACe1 protein.

In order to achieve the above objective, the present application further provides a method for cultivating a plant for controlling Ostrinia furnacalis (Hubern). The method includes the following operations.

At least one plant seed is planted, and a genome of the plant seed comprises a polynucleotide sequence encoding an ACe1 protein.

The plant seed is grown into a plant.

The plant is grown under conditions that the Ostrinia furnacalis (Hubern) is artificially inoculated and/or the hazard of the Ostrinia furnacalis (Hubern) naturally occurs, and a plant that has an attenuated plant damage and/or has an increased plant yield compared with other plants that do not have the polynucleotide sequences encoding the ACe1 protein is harvested.

The “contact” in the present application means that insects and/or pests touch, stay and/or feed on a plant, a plant organ, a plant tissue or a plant cell, and the plant, plant organ, plant tissue or plant cell may be to express the insecticidal protein in vivo, or the plant, plant organ, plant tissue or plant cell has the insecticidal protein on the surface and/or has a microorganism that produces the insecticidal protein.

A term “control” and/or “prevention” in the present application means that the Ostrinia furnacalis (Hubern) is in contact with at least the ACe1 protein, and the growth of the Ostrinia furnacalis (Hubern) is inhibited and/or death is caused after the contact. Further, the Ostrinia furnacalis (Hubern) is in contact with at least the ACe1 protein by ingesting the plant tissue, and after the contact, all or part of the Ostrinia furnacalis (Hubern) is inhibited in growth and/or death is caused. The inhibition refers to sub-lethal, namely it is not lethal but may cause a certain effect in growth, behavior, physiology, biochemistry and tissue and other aspects, such as slow growth and/or stop. At the same time, the plant should be morphologically normal, and may be cultivated by a conventional method for consumption and/or generation of products. In addition, the plant and/or plant seed containing the polynucleotide sequence encoding the ACe1 protein for controlling the Ostrinia furnacalis (Hubern), under the condition that the Ostrinia furnacalis (Hubern) is artificially inoculated and/or the Ostrinia furnacalis (Hubern) naturally occurs, has the reduced plant damage compared with non-transgenic wild plants, and the specific manifestations include, but are not limited to, improved stem resistance, and/or increased grain weight, and/or increased yield, and the like. The “control” and/or “prevention” effect of the ACe1 protein on the Ostrinia furnacalis (Hubern) may exist independently and may not be weakened and/or disappeared due to the presence of other substances that may “control” and/or “prevent” the Ostrinia furnacalis (Hubern). Specifically, if any tissue of the transgenic plant (containing the polynucleotide sequence encoding the ACe1 protein) simultaneously and/or asynchronously exist with and/or produce the ACe1 protein and/or another substance that may control the Ostrinia furnacalis (Hubern), the existence of the another substance neither affects the “control” and/or “prevention” effect of the ACe1 protein on the Ostrinia furnacalis (Hubern), nor may cause the “control” and/or “prevention” effect to be completely and/or partially implemented by the another substance, which is independent of the ACe1 protein. Usually, in the field, the ingestion process of the plant tissue by the Ostrinia furnacalis (Hubern) is short and difficult to observe with naked eyes. Therefore, under the condition that the Ostrinia furnacalis (Hubern) is artificially inoculated and/or the Ostrinia furnacalis (Hubern) naturally occurs, for example, any tissues of the transgenic plant (containing the polynucleotide sequence encoding the ACe1 protein) have the dead Ostrinia furnacalis (Hubern), and/or the Ostrinia furnacalis (Hubern) on which the growth is inhibited, and/or have the reduced plant damage compared with the non-transgenic wild plants, the method and/or the use of the present application is achieved. That is to say, the method and/or the use for controlling the Ostrinia furnacalis (Hubern) is achieved by allowing the Ostrinia furnacalis (Hubern) to be at least in contact with the ACe1 protein.

In the present application, the expression of the ACe1 protein in a transgenic plant may be accompanied by the expression of one or more Cry-like insecticidal proteins and/or Vip-like insecticidal proteins. Co-expression of such more than one insecticidal toxin in the same transgenic plant may be achieved by genetically engineering the plant to contain and express a desired gene. In addition, one plant (first parent) may express the ACe1 protein by a genetic engineering operation, and a second plant (second parent) may express the Cry-like insecticidal proteins and/or Vip-like insecticidal proteins by the genetic engineering operation. Offspring plants expressing all the genes introduced into the first and second parents are obtained by hybridizing the first and second parents.

RNA interference (RNAi) refers to a phenomenon that is highly conserved during the evolution process, and induced by a double-stranded RNA (dsRNA), and a homologous mRNA is efficiently and specifically degraded. Therefore, an RNAi technology may be used in the present application to specifically knock out or shut down the expression of a specific gene in the target insect pest.

The Ostrinia furnacalis (Hubern) in the present application is the most widespread and serious pest in corn production in our country, which is a holometabolous insect belonging to Lepidoptera Pyralidae. Male adults have a body length of 13-14 mm and a wingspan of 22-28 mm; the body back is yellowish-brown; inner wavy transverse lines of the forewings are yellowish-brown, and outer transverse lines are dark brown with zigzag-like patterns. The female adults have a body length of about 14-15 mm and a wingspan of 28-34 mm; and the body is bright yellow with reddish-brown lines. Mature larvae have a body length of 20-30 mm and are in cylindrical shapes; the heads are black-brown; and the back is light gray. In the slightly reddish brown larvae, there are 1 row of 4 circular pinaculums on the metathorax and the back surface; and there are 1 row of 4 circular pinaculums in front of the back of the 1-8 segments of the abdomen, and two in the rear, which are slightly smaller than the front row.

The Ostrinia furnacalis (Hubern) is the most widely distributed and most serious corn pest in China. According to the statistics of the China Agricultural Technology Extension Center, the annual damage area of the pest can reach 350 million mu, and the pest is distributed in the northeast, Huang-huai and southwest regions. Generally, 5-10% loss may be caused, and in severe areas, 30% loss may be caused.

Agrotis ipsilon (Rottemberg) described in the present application is a common agricultural pest, which is an omnivorous insect belonging to Lepidoptera Noctuidae. The pest mainly damages corn, soybeans, cotton, rapes, and the like. The larvae feed on the base of crop stems, resulting in lodging or death, and in severe cases, lack of seedlings and seedlingless ridges may be caused.

Mythimna separate (Walker) described in the present application is a migratory omnivorous pest belonging to Lepidoptera Noctuidae, which damages crops such as corn, rice, sorghum, and the like. The Mythimna separate (Walker) likes to eat leaves. After 3 years old, the pest can bite the whole leaf into a notch shape or eat up heart leaves to form heartless seedlings. When the pest is 5-6 year old, the pest reaches the gluttony period, the pest can eat up all the aerial parts of the seedlings, or eat the entire leaves of the plant, leaving only the veins, resulting in severe yield reduction and even total crop failure.

The ACe1 protein in the present application is a type of β-pore forming protein. The enzymatic cleavage activation in insect gut, receptor binding on the insect gut and a physicochemical environment in the insect gut are key points for achieving the effect of a β-pore forming protein. Only after the β-pore forming protein can be digested into active fragments and bound to the receptor on an epithelial cell membrane of the insect gut, it is possible to make a certain β-pore forming protein have an inhibitory activity against the pests. The receptor binding process requires accurate matching, and often a single amino acid difference in the pore protein or receptor protein can cause changes in binding to the same receptor. For example, after an aerolysin protein belonging to the same β-pore forming protein has qualitative changes in the virulence of a CTLL-2 cell line after R336A mutation (Osusky, Teschk et al, 2008). Likewise, since the receptor is changed, the virulence of the same 3-pore forming protein may also be changed. For example, dsRNA is used to inhibit a HAVCR1 gene in a MDCK cell line, resulting in a hundred-fold difference in the virulence of an epsilon-toxin protein on cells (Ivie, Fennessey et al, 2011). The above fully indicates that the interaction between the β-pore forming protein and enzymes and receptors in insects is complex and unpredictable.

The genome of the plant, plant tissue or plant cell in the present application refers to any genetic materials in the plant, plant tissue or plant cell, and includes a cell nucleus and plastid and mitochondrial genome.

The polynucleotide and/or nucleotide described in the present application forms a complete “gene” that encodes a protein or polypeptide in the required host cell. It is very easily recognized by those skilled in the art that the polynucleotide and/or nucleotide of the present application may be placed under the control of a regulatory sequence in the target host.

It is well-known to those skilled in the art that DNA typically exists in a double-stranded form. In this arrangement, one strand is complementary to the other strand and vice versa. Other complementary strands of DNA are produced as a result of DNA replication in the plants. In this way, the present application includes the use of the polynucleotide exemplified in a sequence listing and complementary strands thereof. A “coding strand” as commonly used in the field refers to a strand that binds to an antisense strand. In order to express a protein in vivo, typically one strand of DNA is transcribed into a complementary strand of mRNA, it serves as a template for translation of the protein. mRNA is actually transcribed from the “antisense” strand of DNA. The “sense” or “coding” strand has a series of codons (the codon is three nucleotides, and a specific amino acid may be produced by reading three at a time), and it may be read as an open reading frame (OR F) to form a target protein or peptide. The present application also includes RNA that is functionally equivalent to the exemplified DNA.

The nucleic acid molecule or fragment thereof in the present application hybridizes to the ACe1 gene of the present application under stringent conditions. Any conventional nucleic acid hybridization or amplification methods may be used to identify the presence of the ACe1 gene of the present application. The nucleic acid molecule or fragment thereof is capable of specifically hybridizing with other nucleic acid molecules under certain circumstances. In the present application, if two nucleic acid molecules may form an anti-parallel double-stranded nucleic acid structure, it may be said that the two nucleic acid molecules may specifically hybridize with each other. If the two nucleic acid molecules show complete complementarity, one nucleic acid molecule is said to be a “complement” of the other nucleic acid molecule. In the present application, while each nucleotide of one nucleic acid molecule is complementary to the corresponding nucleotide of the other nucleic acid molecule, the two nucleic acid molecules are said to show the “complete complementarity”. If the two nucleic acid molecules may hybridize to each other with sufficient stability such that they anneal and bind to each other under at least conventional “low stringency” conditions, the two nucleic acid molecules are said to be “minimally complementary”. Similarly, if the two nucleic acid molecules may hybridize to each other with the sufficient stability such that they anneal and bind to each other under conventional “high stringency” conditions, the two nucleic acid molecules are said to have “complementarity”. Deviation from the complete complementarity is permissible as long as such deviation does not completely prevent the two molecules from forming the double-stranded structure. In order for a nucleic acid molecule to function as a primer or a probe, it only needs to be sufficiently complementary in its sequence, as to allow for the formation of the stable double-stranded structure under adopted particular solvent and salt concentration.

In the present application, the substantially homologous sequence is a section of a nucleic acid molecule, the nucleic acid molecule may specifically hybridize with a complementary strand of another matched nucleic acid molecule under highly stringent conditions. Suitable stringent conditions to promote the DNA hybridization, for example, treatment with 6.0×sodium chloride/sodium citrate (SSC) at about 45° C., and followed by washing with 2.0×SSC at 50° C., are well-known to those skilled in the art. For example, the salt concentration in a washing step may be selected from about 2.0×SSC and 50° C. under the low stringency conditions to about 0.2×SSC and 50° C. under the high stringency conditions. In addition, the temperature condition in the washing step may be increased from about 22° C. at a room temperature under the low stringency conditions to about 65° C. under the high stringency conditions. Both the temperature condition and the salt concentration may be changed, or one of which may be kept unchanged while the other variable is changed. Preferably, the stringency condition described in the present application may be specific hybridization in 6×SSC and 0.5% sodium dodecyl sulfate (SDS) solutions at 65° C., and then membrane-washing once with 2×SSC, 0.1% SDS and 1×SSC and 0.1% SDS.

Therefore, sequences that have the insecticidal activity and hybridize to SEQ ID NO:19 to SEQ ID NO:36 of the present application under the stringency condition are included in the present application. These sequences have at least about 40%-50% of the identity with the sequences of the present application, about 60%, 65% or 70% of the identity, even at least about 75%, 80%, 85%, 90%, 91%, 92%, 93%, 94%, 95%, 96%, 97%, 98%, 99% or greater sequence identity.

The genes and proteins described in the present application include not only a specific exemplified sequence, but also include parts and/or fragments (including internal and/or terminal deletion as compared with a full-length protein) that preserve the characteristics of the insecticidal activity of the specific exemplified protein, a variant, a mutant, a substitute (a protein with a substituted amino acid), a chimera and a fusion protein. The “variant” or “variation” refers to a nucleotide sequence encoding the same protein or encoding an equivalent protein with the insecticidal activity. The “equivalent protein” refers to a protein that has the same or substantially the same biological activity against the Ostrinia furnacalis (Hubern) as the claimed protein.

A “fragment” or “truncation” of the DNA molecule or protein sequence described in the present application refers to a portion of the original DNA or protein sequence (nucleotide or amino acid) involved or an artificially modified form thereof (for example, a sequence suitable for plant expression), the length of the aforementioned sequence may have a change but is long enough to ensure that the (encoded) protein is an insect toxin.

A standard technology may be used to modify the gene and construct the genetic variant easily, for example, a technology for manufacturing a point mutation which is well-known in the field. As another example, U.S. Pat. No. 5,605,793 describes a method for producing additional molecular diversity using DNA reassembly after random fragmentation. The fragment of the full-length gene may be manufactured with a commercial endonuclease, and an exonuclease may be used according to a standard procedure. For example, enzymes such as Bal31 or site-directed mutagenesis may be used to systematically excise nucleotides from the ends of these genes. The genes encoding the active fragments may also be obtained with a plurality of restriction enzymes. The active fragments of these toxins may be obtained directly with proteases.

The present application may derive equivalent proteins and/or genes encoding these equivalent proteins from a β-pore forming protein isolate and/or a DNA library. There are various ways to obtain the insecticidal protein of the present application. For example, antibodies of the insecticidal protein disclosed and claimed in the present application may be used to identify and isolate other proteins from protein mixtures. In particular, the antibodies may arise from a portion of the protein that is most constant and most different from other β-pore forming proteins. These antibodies may then be used to specifically identify the equivalent proteins with the characteristic activity by immunoprecipitation, an enzyme-linked immunosorbent assay (ELISA), or a western blotting method. Antibodies of the proteins or the equivalent proteins or the fragments of such proteins disclosed in the present application may be easily prepared by the standard procedure in the field. The genes encoding these proteins may then be obtained from the microorganisms.

Due to the redundancy of genetic codons, many different DNA sequences may encode the same amino acid sequence. The generation of these alternative DNA sequences encoding the same or substantially same protein is within the technological level of those skilled in the art. These various DNA sequences are included within a scope of the present application. The “substantially same” sequence refers to a sequence with amino acid substitution, deletion, addition or insertion that does not substantially affect the insecticidal activity, and also includes a fragment that retains the insecticidal activity.

The substitution, deletion or addition of the amino acid sequence in the present application is a routine technology in the field, preferably such an amino acid change is: a small property change, namely conservative amino acid substitution that does not significantly affect the folding and/or activity of the protein; small deletion, typically deletion of about 1-30 amino acids; small amino- or carboxy-terminal extension, for example, amino-terminal extension of one methionine residue; and a small linker peptide, for example, the length of about 20-25 residues.

Examples of the conservative substitution are those that occur within the following amino acid groups: basic amino acids (such as an arginine, a lysine, and a histidine), acidic amino acids (such as a glutamic acid and an aspartic acid), polar amino acids (such as a glutamine, and an asparagine), hydrophobic amino acids (such as a leucine, an isoleucine, and a valine), aromatic amino acids (such as a phenylalanine, a tryptophan, and a tyrosine), and small molecular amino acids (such as a glycine, an alanine, a serine, a threonine, and a methionine). Those amino acid substitutions that generally do not change the specific activity are well-known in the field, and already described, for example, by N. Neurath and R. L. Hill in “Protein” published by Academic Press, New York in 1979. The most common interchanges are Ala/Ser, Val/Ile, Asp/Glu, Thu/Ser, Ala/Thr, Ser/Asn, AlaNal, Ser/Gly, Tyr/Phe, Ala/Pro, Lys/Arg, Asp/Asn, Leu/Ile, LeuNal, Ala/Glu and Asp/Gly, and their opposite interchanges.

It is apparent to those skilled in the art that such substitutions may occur outside areas important to the function of the molecule, and still produce the active polypeptide. The amino acid residues that are essential for the activity of the polypeptide of the present application and are therefore selected not to be substituted may be identified according to methods known in the field, such as site-directed mutagenesis or alanine-scanning mutagenesis (referring to, for example, Cunningham and Wells, 1989, Science 244: 1081-1085). The latter technology is to introduce a mutation at each positively charged residue in the molecule, and to test the inhibitory activity of the mutant molecules obtained, thereby the amino acid residues that are important to the activity of the molecule are determined. Substrate-enzyme interaction sites may also be determined by analysis of its three-dimensional structure, this three-dimensional structure may be determined by technologies such as nuclear magnetic resonance analysis, crystallography, or photoaffinity labeling (referring to, for example, de Vos et al., 1992, Science 255:306-312; Smith et al., 1992, J. Mol. Biol 224:899-904; and Wodaver et al., 1992, FEBS Letters 309:59-64).

In the present application, the ACe1 protein includes, but is not limited to, SEQ ID NO:1 to SEQ ID NO:18, and amino acid sequences having certain identity with the amino acid sequences shown in SEQ ID NO:1 to SEQ ID NO:18 are also included in the present application. These sequences are typically greater than 78% of similarity/identity of the sequence of the present application, preferably greater than 85%, more preferably greater than 90%, even more preferably greater than 95%, and may be greater than 99%. Preferred polynucleotides and proteins of the present application may also be defined according to more specific ranges of the identity and/or similarity. For example, there are 78%, 79%, 80%, 81%, 82%, 83%, 84%, 85%, 86%, 87%, 88%, 89%, 90%, 91%, 92%, 93%, 94%, 95%, 96%, 97%, 98% or 99% of the identity and/or similarity with the sequences exemplified in the present application.

The regulatory sequence described in the present application includes, but is not limited to, a promoter, a transit peptide, a terminator, an enhancer, a leader sequence, an intron, and other regulatory sequences operably linked to the ACe1 protein.

The promoter is a promoter expressible in the plant, and the “promoter expressible in the plant” refers to a promoter that ensures the expression of the coding sequence linked to it in plant cells. The promoter expressible in the plant may be a constitutive promoter. Examples of the promoter that direct the constitutive expression in the plant include, but are not limited to, a 35S promoter derived from a cauliflower mosaic virus, a maize Ubi promoter, a promoter of a rice GOS2 gene and the like. Alternatively, the promoter expressible in the plant may be a tissue-specific promoter, namely the promoter directs the expression of the coding sequence to a higher level in some tissues of the plant, such as in a green tissue, than in other tissues of the plant (may be determined by a conventional RNA test), such as a PEP carboxylase promoter. Alternatively, the promoter expressible in the plant may be a wound-inducible promoter. The wound-inducible promoter or a promoter that directs a wound-induced expression pattern means that the expression of the coding sequence under the control of the promoter is significantly increased while the plant is subjected to a mechanical or insect-induced wound compared to normal growth conditions. Examples of the wound-inducible promoter include, but are not limited to, promoters of protease inhibitory genes (pin I and pin II) of potato and tomato and a promoter of a maize protease inhibitor gene (MPI). Examples of the wound-inducible promoter include but are not limited to promoters of protease inhibitory genes (pin I and pin II) of potato and tomato and a promoter of a maize protease inhibitor gene (MPI).

The transit peptide (also known as a secretion signal sequence or a targeting sequence) directs a transgenic product to a specific organelle or cellular compartment, the transit peptide may be heterologous to the receptor protein, for example, with a transit peptide sequence encoding a chloroplast to target the chloroplast or using a ‘KDEL’ retention sequence to target an endoplasmic reticulum or using CTPP of a barley lectin gene to target a vacuole.

The leader sequence includes, but is not limited to, a picornavirus leader sequence, such as an encephalomyocarditis virus 5′ non-coding region (EMCV) leader sequence; a potato Y virus group leader sequence, such as a maize dwarf mosaic virus (MDMV) leader sequence; a human immunoglobulin heavy chain binding protein (BiP); an untranslated leader sequence of coat protein mRNA of alfalfa mosaic virus (AMV RNA4); and a tobacco mosaic virus (TMV) leader sequence.

The enhancer includes, but is not limited to, a cauliflower mosaic virus (CaMV) enhancer, a figwort mosaic virus (FMV) enhancer, a carnation etched ring virus (CERV) enhancer, a cassava vein mosaic virus (CsVMV) enhancer, a mirabilis mosaic virus (MMV) enhancer, a cestrum yellow leaf curling virus (CmYLCV) enhancer, a cotton leaf curl multan virus (CLCuMV), a commellna yellow motile virus (CoYMV) and a peanut chlorella leaf strip virus (PCLSV) enhancer.

For monocot applications, the intron includes, but is not limited to, a maize hsp70 intron, a maize ubiquitin intron, an Adh intron 1, a sucrose synthase intron, or a rice Act1 intron. For dicot applications, the intron includes, but is not limited to, a CAT-1 intron, a pKANNIBAL intron, a PIV2 intron, and a “super ubiquitin” intron.

The terminator may be a suitable polyadenylation signal sequence functioning in the plant, including, but not limited to, a polyadenylation signal sequence derived from a nopaline synthase (NOS) gene of Agrobacterium tumefaciens, a polyadenylation signal sequence derived from a protease inhibitor II (pin II) gene, a polyadenylation signal sequence derived from a pea ssRUBISCO E9 gene, and a polyadenylation signal sequence derived from a α-tubulin gene.

The “operably linked” in the present application refers to association of nucleic acid sequences such that one sequence may provide a desired function for the linked sequence. In the present application, the “operably linked” may be to link a promoter with an interested sequence, so that the transcription of the interested sequence is controlled and regulated by the promoter. While the interested sequence encodes a protein and the expression of the protein is desired, the “operably linked” means that: the promoter is linked to the sequence, so that an obtained transcript is efficiently translated in a linkage mode. If the linkage of the promoter to the coding sequence is transcript fusion and the expression of the encoded protein is desired, such linkage is manufactured, so that the first translation initiation codon in the obtained transcript is an initiation codon of the coding sequence. Alternatively, if the linkage of the promoter to the coding sequence is translational fusion and the expression of the encoded protein is desired, such linkage is manufactured, so that the first translation initiation codon contained in a 5′ untranslated sequence is linked to the promoter, and a relationship between an obtained translation product and a translational open reading frame encoding the desired protein accords with the reading frame in the linkage mode. The nucleic acid sequence that may be “operably linked” includes, but is not limited to: sequences providing gene expression functions (namely gene expression elements such as a promoter, a 5′ untranslated region, an intron, a protein coding region, a 3′ untranslated region, a poly adenylation site and/or a transcription terminator), sequences providing DNA transfer and/or integration functions (namely a T-DNA border sequence, a site-specific recombinase recognition site, and an integrase recognition site), sequences providing selectivity functions (namely an antibiotic resistance marker, and a biosynthetic gene), sequences providing scoreable marker functions, sequences that assist in sequence operation in vitro or in vivo (namely a polylinker sequence, and a site-specific recombination sequence) and sequences providing replication functions (namely a bacterial replication origin, a autonomously replicating sequence, and a centromeric sequence).

In the present application, the “insecticide” or “insect resistance” means that it is toxic to crop pests, thereby the “control” and/or “prevention” of the crop pests is achieved. Preferably, the “insecticide” or “insect resistance” means that the crop pests are killed. More specifically, the target insect is the Ostrinia furnacalis (Hubern).

The ACe1 protein in the present application is virulent to the Ostrinia furnacalis (Hubern). The plant in the present application, especially the corn, contains an exogenous DNA in its genome. The exogenous DNA contains a nucleotide sequence encoding the ACe1 protein. The Ostrinia furnacalis (Hubern) is in contact with the protein by ingesting the plant tissue, and after the contact, the growth of the Ostrinia furnacalis (Hubern) is inhibited and/or death is caused. The inhibition means lethal or sub-lethal. At the same time, the plant should be morphologically normal, and may be cultivated under a conventional method for consumption and/or generation of products. In addition, the plant may substantially eliminate the need for a chemical or biological pesticide (the chemical or biological pesticide is an insecticide against the Ostrinia furnacalis (Hubern) targeted by the ACe1 protein).

The expression level of an insecticidal protein in the plant material may be detected by a plurality of methods described in the field, for example, by applying a specific primer to quantify mRNA encoding the insecticidal protein produced in the tissue, or directly specifically detecting the amount of the insecticidal protein produced.

Different tests may be applied to determine the insecticidal effect of the insecticidal protein in the plant. In the present application, the target insect is mainly the Ostrinia furnacalis (Hubern).

In the present application, the ACe1 protein may have the amino acid sequences shown in SEQ ID NO:1 to SEQ ID NO:18 in the sequence listing. In addition to the coding region containing the ACe1 protein, other elements may also be included, such as a protein encoding a selectable marker.

In addition, an expression cassette containing the polynucleotide sequence encoding the ACe1 protein of the present application may also be expressed in the plant together with at least one protein encoding a herbicide resistance gene, the herbicide resistance gene includes, but not limited to, a glufosinate-ammonium resistance gene (such as a bar gene, and a pat gene), a Betanal resistance gene (such as a pmph gene), a glyphosate resistance gene (such as an EPSPS gene), a bromoxynil resistance gene, a sulfonylurea resistance gene, an anti-herbicide dalapon resistance gene, an anti-cyanamide resistance gene or a resistance gene of a glutamine synthase inhibitor (such as PPT), as to obtain the transgenic plant having both high insecticidal activity and herbicide resistance.

In the present application, the exogenous DNA is introduced into the plant, for example, the gene or expression cassette or recombinant vector encoding the ACe1 protein is introduced into the plant cell, and the conventional transformation method includes, but not limited to, agrobacterium-mediated transformation, micro-emission bombardment, direct DNA ingestion into a protoplast, electroporation, or whisker silicon-mediated DNA introduction.

The present application provides a use of an insecticidal protein and has the following advantages.

1. Prevention and treatment of internal causes: The prior art mainly controls the harm of the Ostrinia furnacalis (Hubern) by the external action namely the external causes, for example, the agricultural control, the chemical control, the physical control and the biological control; and the present application controls the Ostrinia furnacalis (Hubern) by producing the ACe1 protein that may kill the Ostrinia furnacalis (Hubern) in the plant, namely the Ostrinia furnacalis (Hubern) is controlled by the internal causes.

2. No pollution and no residue: Although the chemical control method used in the prior art plays a certain role in controlling the harm of the Ostrinia furnacalis (Hubern), it also brings the pollution, damage and residue to humans, livestocks and farmland ecosystems; and with the method of the present application to control the Ostrinia furnacalis (Hubern), the above adverse consequences may be eliminated.

3. Prevention and control during whole growth period: The methods used in the prior art to control the Ostrinia furnacalis (Hubern) are all staged, and the present application is to protect the plant during the whole growth period, and the transgenic plant (ACe1 protein) may be prevented from being attacked by the Ostrinia furnacalis (Hubern) from germination, growth, to flowering and fruiting.

4. Whole plant control: Most of the methods used in the prior art to control the Ostrinia furnacalis (Hubern) are localized, such as foliar spraying; and the present application protects the entire plant, for example, roots, leaves, stems, fruits, tassels, female ears, anthers or filaments of the transgenic plant (ACe1 protein) are all resistant to the attack of the Ostrinia furnacalis (Hubern).

5. Stable effect: Whether it is the agricultural control method or the physical control method used in the prior art, it is necessary to use the environmental conditions to control the pests, and there are many variable factors; the present application is to express the ACe1 protein in the plant, which effectively overcomes the disadvantages of the unstable environmental conditions, and the control effect of the transgenic plant (ACe1 protein) of the present application is stable and consistent in different places, different times and different genetic backgrounds.

6. Simpleness, convenience and economy: The present application only needs to plant the transgenic plant capable of expressing the ACe1 protein, and does not need to adopt other measures, thereby reducing a lot of manpower, material resources and financial resources.

7. Complete effect: The methods used in the prior art to control the Ostrinia furnacalis (Hubern) are not thorough in effect, and only play a role in relieving; and the transgenic plant (ACe1 protein) of the present application may cause a large number of deaths of the newly hatched larvae of the Ostrinia furnacalis (Hubern).

The technical schemes of the present application are further described in detail below by drawings and embodiments.

### BRIEF DESCRIPTION OF THE DRAWINGS

FIG. 1 is a construction flowchart of a recombinant expression vector DBN01-P containing an ACe1 nucleotide sequence for the use of the insecticidal protein according to the present application.

### DETAILED DESCRIPTION OF THE EMBODIMENTS

The technical schemes of the use of the insecticidal protein of the present application are further described below by specific embodiments.

### Example 1: Acquisition and Synthesis of Gene

1. Acquisition of the Nucleotide Sequence

An amino acid sequence of an ACe1 insecticidal protein is shown in Table 1; and an ACe1 nucleotide sequence encoding the amino acid sequence corresponding to the ACe1 insecticidal protein is shown in Table 1.

TABLE 1
ACe1 protein and corresponding amino acid and
nucleotide sequence thereof

| Insecticidal protein | Amino acid | Bacterial nucleotide | Plant nucleotide |
|---|---|---|---|
| name | SEQ ID NO. | SEQ ID NO. | SEQ ID NO. |
| ACe1_3 | SEQ ID NO: 1 | SEQ ID NO: 19 | SEQ ID NO: 43 |
| ACe1_4 | SEQ ID NO: 2 | SEQ ID NO: 20 | SEQ ID NO: 44 |
| ACe1_5 | SEQ ID NO: 3 | SEQ ID NO: 21 | SEQ ID NO: 45 |
| ACe1_6 | SEQ ID NO: 4 | SEQ ID NO: 22 | SEQ ID NO: 46 |
| ACe1_8 | SEQ ID NO: 5 | SEQ ID NO: 23 | SEQ ID NO: 47 |
| ACe1_9 | SEQ ID NO: 6 | SEQ ID NO: 24 | SEQ ID NO: 48 |
| ACe1_10 | SEQ ID NO: 7 | SEQ ID NO: 25 | SEQ ID NO: 49 |
| ACe1_11 | SEQ ID NO: 8 | SEQ ID NO: 26 | SEQ ID NO: 50 |
| ACe1_12 | SEQ ID NO: 9 | SEQ ID NO: 27 | SEQ ID NO: 51 |
| ACe1_13 | SEQ ID NO: 10 | SEQ ID NO: 28 | SEQ ID NO: 52 |
| ACe1_14 | SEQ ID NO: 11 | SEQ ID NO: 29 | SEQ ID NO: 53 |
| ACe1_15 | SEQ ID NO: 12 | SEQ ID NO: 30 | SEQ ID NO: 54 |
| ACe1_16 | SEQ ID NO: 13 | SEQ ID NO: 31 | SEQ ID NO: 55 |
| ACe1_17 | SEQ ID NO: 14 | SEQ ID NO: 32 | SEQ ID NO: 56 |
| ACe1_18 | SEQ ID NO: 15 | SEQ ID NO: 33 | SEQ ID NO: 57 |
| ACe1_19 | SEQ ID NO: 16 | SEQ ID NO: 34 | SEQ ID NO: 58 |
| ACe1_20 | SEQ ID NO: 17 | SEQ ID NO: 35 | SEQ ID NO: 59 |
| ACe1_21 | SEQ ID NO: 18 | SEQ ID NO: 36 | SEQ ID NO: 60 |

2. Synthesis of Above Nucleotide Sequence

Nucleotide sequences (as shown in SEQ ID NO:19 to SEQ ID NO:36 in the sequence listing) of the above 18 ACe1 proteins are synthesized by Nanjing Genscript Biotechnology Co., Ltd.

### Example 2: Construction of Recombinant Expression Vector and Transformation of Recombinant Expression Vector into Escherichia coli to Obtain an ACe1 Protein

1. Construction of Recombinant Expression Vector Containing an ACe1 Gene

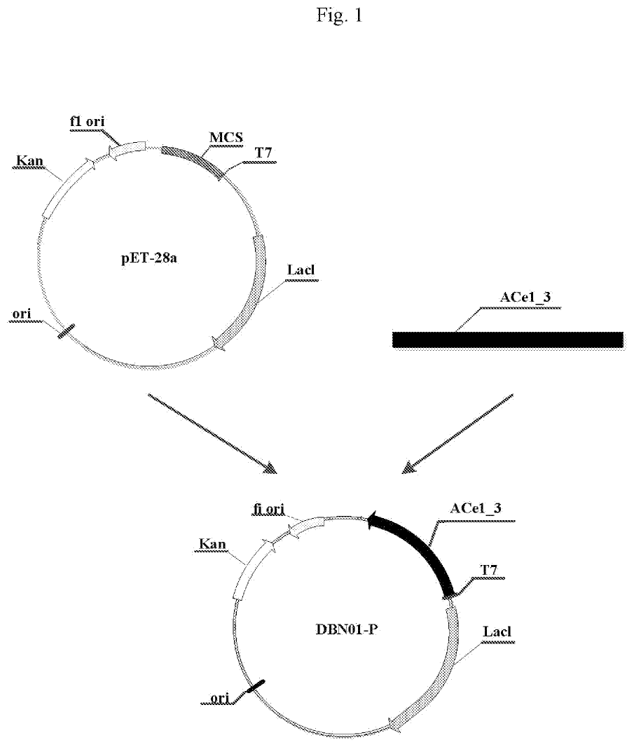

The nucleotide sequences of the ACe1 proteins (ACe1_3 to ACe1_6, ACe1_8 to ACe1_21) synthesized in the Example 1 are linked into a protein expression vector pET28a (Novagen, USA, CAT: 69864-3); operation steps are performed according to the specification of the product pET28a vector of Novagen, so as to obtain recombinant expression vectors DBN01-P to DBN18-P; and a construction flow is shown in FIG. 1 (where Kan represents a kanamycin resistance gene, f1 ori represents the origin of replication of phage f1, Lad is a Lad initiation codon, ACe1_3 is an ACe1_3 nucleotide sequence (SEQ ID NO:19), and MCS represents multiple cloning sites).

The ACe1 protein and names of the corresponding recombinant expression vectors thereof are shown in Table 2.

TABLE 2
ACe1 protein and names of corresponding
recombinant expression vectors thereof

| Insecticidal protein name | Recombinant expression vector |
|---|---|
| ACe1_3 | DBN01-P |
| ACe1_4 | DBN02-P |
| ACe1_5 | DBN03-P |
| ACe1_6 | DBN04-P |
| ACe1_8 | DBN05-P |
| ACe1_9 | DBN06-P |
| ACe1_10 | DBN07-P |
| ACe1_11 | DBN08-P |
| ACe1_12 | DBN09-P |
| ACe1_13 | DBN10-P |
| ACe1_14 | DBN11-P |
| ACe1_15 | DBN12-P |
| ACe1_16 | DBN13-P |
| ACe1_17 | DBN14-P |
| ACe1_18 | DBN15-P |
| ACe1_19 | DBN16-P |
| ACe1_20 | DBN17-P |
| ACe1_21 | DBN18-P |

2. Transformation of Recombinant Expression Vector into Escherichia coli to Obtain ACe1 Protein

Then, the recombinant expression vectors DBNO1 to DBN18-P are transformed into Escherichia coli BL21(DE3) competent cells (Transgen, China, CAT: CD501) by a heat shock method; positive colonies are picked and placed in an LB liquid medium (10 g/L of a tryptone, 5 g/L of a yeast extract, 10 g/L of NaCl, 100 mg/L of an ampicillin, and pH is adjusted to 7.5 with NaOH); and cultured for 16 h at 37° C. and at 200 r/min. The culture solution is then transferred to an YT culture medium according to the proportion of 1:10; and culture is performed at 37° C. and at 200 r/min. When an OD=600 value of the culture solution reaches 0.6-0.8, IPTG is added until a final concentration is 0.5 mM, so as to perform inducible expression for 6h, and the culture solution is centrifuged to collect the cells; the supernatant is discarded, resuspending is performed after PBS is added, and then ultrasonic disruption is performed; and the expression protein is detected by SDS-PAGE, the protein concentration is estimated, and preservation is performed at −20° C. for later use.

### Example 3, Identification of Inhibitory Activity Against Ostrinia furnacalis (Hubern) by Feeding the ACe1 Protein

Inhibitory activity against the Ostrinia furnacalis (Hubern), the Agrotis ipsilon (Rottemberg) and the Mythimna separate (Walker) is detected with the series of ACe1 proteins (ACe1_3 to ACe1_6, ACe1_8 to ACe1_21) obtained in part 2 of Example 2. A total of 18 treatments are designed for each pest, which respectively are ACe1_3 to ACe1_6, ACe1_8 to ACe1_21; and 1 negative control treatment is designed, which is GFP. Protein liquid of ACe1_3 to ACe1_6, ACe1_8 to ACe1_21, and GFP are respectively mixed in feed, and a final concentration is 50 μg/g. Each group of treatments is repeated for 3 times.

TABLE 3
Inhibitory activity results of Ostrinia furnacalis (Hubern),
Agrotis ypsilon (Rottemberg) and Mythimna separate
(Walker) that are fed with the ACe1 protein

| Serial | | Test insect | Mythimna |
|---|---|---|---|
| number | Ostrinia furnacalis | Agrotis ypsilon | separate |
| of proteins | (Hubern) | (Rottemberg) | (Walker) |
| ACe1_3 | + | S | S |
| ACe1_4 | + | S | S |
| ACe1_5 | + | S | S |
| ACe1_6 | + | S | S |
| ACe1_8 | + | S | S |
| ACe1_9 | + | S | S |
| ACe1_10 | + | S | S |
| ACe1_11 | + | S | S |
| ACe1_12 | − | − | − |
| ACe1_13 | + | S | S |
| ACe1_14 | + | S | S |
| ACe1_15 | − | − | − |
| ACe1_16 | + | S | S |
| ACe1_17 | + | S | S |
| ACe1_18 | + | S | S |
| ACe1_19 | − | − | − |
| ACe1_20 | − | − | − |
| ACe1_21 | − | − | − |
| GFP | − | − | − |

“+” represents that there is a lethal effect;
“−” represents that there is no inhibitory activity;
″NT″ stands for not tested; and
″S″ represents developmental inhibition

Results in Table 3 show that, ACe1_3 to ACe1_6, ACe1_8 to ACe1_11, ACe1_13, ACe1_14, ACe1_16 to ACe1_18 proteins show desirable inhibitory activity against the Ostrinia furnacalis (Hubern), and only show the effect of developmental inhibition on the Agrotis ipsilon (Rottemberg) and the Mythimna separate (Walker).

Therefore, it indicates that the ACe1 proteins (ACe1_3 to ACe1_6, ACe1_8 to ACe1_11, ACe1_13, ACe1_14, ACe1_16 to ACe1_18) show resistance activity against the Ostrinia furnacalis (Hubern), and this activity is sufficient to have adverse effects on the growth of the Ostrinia furnacalis (Hubern), so that the Ostrinia furnacalis (Hubern) can be controlled in the fields. In addition, it is also possible to reduce the occurrence of diseases on the transgenic ACe1 plants by controlling the damage of the Ostrinia furnacalis (Hubern), thereby greatly improving the yield and quality of the transgenic ACe1 plants.

In conclusion, through the use of the insecticidal protein of the present application, ACe1 protein that can kill the Ostrinia furnacalis (Hubern) is produced in a plant body to control the Ostrinia furnacalis (Hubern). Compared with an agricultural control method, a chemical control method, a physical control method and a biological control method used in the prior art, the present application achieves the protection of whole growth period and whole plant on the plants so as to control the infestation of the Ostrinia furnacalis (Hubern), and is pollution-free, residue-free, stable in effect, thorough, simple, convenient and economical.

Finally, it should be noted that the above embodiments are only used to illustrate the technical schemes of the present application and not to limit them. Although the present application is described in detail with reference to the preferred embodiments, those of ordinary skill in the art should understand that the technical schemes of the present application may be modified or equivalently replaced without departing from the spirit and scope of the technical schemes of the present application.

What is claimed is:

1. A method for controlling Ostrinia furnacalis (Hubern), comprising allowing the Ostrinia furnacalis (Hubern) to be at least in contact with an ACe1 protein;
preferably, the ACe1 protein is present in a host cell that produces at least the Ace1 protein, and the Ostrinia furnacalis (Hubern) is in contact with at least the Ace1 protein by ingesting the host cell; and
more preferably, the Ace1 protein is present in bacteria or a transgenic plant that produces at least the ACe1 protein, the Ostrinia furnacalis (Hubern) is in contact with at least the ACe1 protein by ingesting the bacterium or a tissue of the transgenic plant, and after contacting, the growth of the Ostrinia furnacalis (Hubern) is inhibited and/or death is caused, so as to achieve the control of the damage of the Ostrinia furnacalis (Hubern) to plants.

2. The method for controlling Ostrinia furnacalis (Hubern) according to claim 1, wherein the transgenic plant is corn or sorghum.

3. The method for controlling Ostrinia furnacalis (Hubern) according to claim 1, wherein the tissue of the transgenic plant is a root, a leaf, a stem, a tassel, an ear, an anther, or a filament.

4. The method for controlling Ostrinia furnacalis (Hubern) according to claim 1, wherein the ACe1 protein is an ACe1_3 protein, an ACe1_4 protein, an ACe1_5 protein, ACe1_6 protein, an ACe1_8 protein, an ACe1_9 protein, an ACe1_10 protein, an ACe1_11 protein, an ACe1_12 protein, an ACe1_13 protein, an ACe1_14 protein, an ACe1_15 protein, an ACe1_16 protein, an Ace1_17 protein, an ACe1_18 protein, an ACe1_19 protein, an ACe1_20 protein, or an ACe1_21 protein.

5. The method for controlling Ostrinia furnacalis (Hubern) according to claim 4, wherein the ACe1 protein has an amino acid sequence shown in any one of SEQ ID NO:1 to SEQ ID NO:18.

6. The method for controlling Ostrinia furnacalis (Hubern) according to claim 4, wherein the ACe1 protein has an amino acid sequence shown in any one of SEQ ID NO:19 to SEQ ID NO: 36; and the ACe1 protein has a nucleotide sequence in the transgenic plant that are shown in any one of SEQ ID NO:43 to SEQ ID NO:60.

7. The method for controlling Ostrinia furnacalis (Hubern) according to claim 6, wherein the transgenic plant further comprises at least one second nucleotide different from the nucleotide encoding the ACe1 protein.

8. The method for controlling Ostrinia furnacalis (Hubern) according to claim 7, wherein the second nucleotide encodes a Cry-like insecticidal protein, a Vip-like insecticidal protein, a protease inhibitor, lectin, α-amylase, or a peroxidase.

9. The method for controlling Ostrinia furnacalis (Hubern) according to claim 7, wherein the second nucleotide encodes a Cry1Ab protein, a Cry2Ab protein, or a Cry1A.105 protein.

10. The method for controlling Ostrinia furnacalis (Hubern) according to claim 9, wherein the Cry1Ab protein, the Cry2Ab protein, or the Cry1A.105 protein has an amino acid sequence shown in SEQ ID NO:37, SEQ ID NO:38, or SEQ ID NO:39, respectively.

11. The method for controlling Ostrinia furnacalis (Hubern) according to claim 7, wherein the second nucleotide has a nucleotide sequence shown in SEQ ID NO:40, SEQ ID NO:41, or SEQ ID NO:42.

12. The method for controlling Ostrinia furnacalis (Hubern) according to claim 7, wherein the second nucleotide is a dsRNA that inhibits an important gene in a target insect pest.

13. A method of producing a plant for controlling Ostrinia furnacalis (Hubern), comprising introducing a polynucleotide sequence encoding an ACe1 protein into a genome of the plant.

14. The method of producing a plant for controlling Ostrinia furnacalis (Hubern) according to claim 13, wherein the polynucleotide sequence of the ACe1 protein is shown in any one of SEQ ID NO:43 to SEQ ID NO:60.

15. The method of producing a plant for controlling Ostrinia furnacalis (Hubern) according to claim 13, wherein the ACh1 protein has an amino acid sequence shown in any one of SEQ ID NO:19 to SEQ ID NO: 36.

16. A method of producing a plant seed for controlling Ostrinia furnacalis (Hubern), comprising hybridizing a first plant obtained by the method according to claim 13 with a second plant, so as to produce a seed containing a polynucleotide sequence encoding an ACe1 protein.

17. A method of cultivating a plant for controlling Ostrinia furnacalis (Hubern), comprising:
planting at least one plant seed, wherein a genome of the plant seed comprises a polynucleotide sequence encoding an ACe1 protein;
growing the plant seed into a plant; and
growing the plant under conditions that the Ostrinia furnacalis (Hubern) is artificially inoculated and/or the hazard of the Ostrinia furnacalis (Hubern) naturally occurs, and harvesting a plant that has an attenuated plant damage and/or has an increased plant yield compared with other plants that do not have the polynucleotide sequences encoding the ACe1 protein.

18. The method of cultivating a plant for controlling Ostrinia furnacalis (Hubern) according to claim 17, wherein the polynucleotide sequence of the ACe1 protein is shown in any one of SEQ ID NO:43 to SEQ ID NO:60.

19. The method of cultivating a plant for controlling Ostrinia furnacalis (Hubern) according to claim 17, wherein the ACh1 protein has an amino acid sequence shown in any one of SEQ ID NO:19 to SEQ ID NO: 36.
